(19) United States
(12) Patent Application Publication
(10) Pub. No.: US 2009/0004131 A1
(43) Pub. Date: Jan. 1, 2009

(54) WAX-FREE COSMETIC COMPOSITION IN FOAM FORM

(75) Inventors: Patrice STYCZEN, Gif-Sur-Yvette (FR); Xavier Ray, Villeconin (FR); Philippe Gabin, Bures Sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/113,969

(22) Filed: May 2, 2008

Related U.S. Application Data
(60) Provisional application No. 60/939,666, filed on May 23, 2007.

(30) Foreign Application Priority Data
May 10, 2007 (FR) 07 54977

(51) Int. Cl. A61K 8/89; A61Q 5/00; A61Q 1/00

(52) U.S. Cl. 424/70.122; 424/70.12; 424/78.03

(57) ABSTRACT

A composition in the foam form having a continuous oily phase and at least one structuring agent for the oily phase, the composition being devoid of wax. Method of use. Kit.

### REFERENCE TO PRIOR APPLICATIONS

This application claims priority to U.S. provisional application 60/939,666 filed May 23, 2007, and to French patent application 0754977 filed May 10, 2007, both incorporated herein by reference.

### FIELD OF THE INVENTION

The present invention relates to a composition in the foam form comprising a continuous oily phase.

The compositions according to the invention can be used for, e.g., making up or caring for keratinous substances, in particular the skin, lips, eyelashes, eyebrows or nails. The composition may or may not be coloured. The compositions according to the invention can be, e.g., a foundation, a face powder, an eye shadow, a concealer, a blusher, a lipstick, a lip balm, a lip gloss, a mascara, an eyeliner or a product for making up the body or colouring the skin. They can also be, e.g., care compositions such as products for caring for the eyelashes, lips or skin of the body and face, in particular an antisun product.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Compositions in the foam form which are oil-in-water or wax-in-water emulsions comprising a continuous aqueous phase are known. However, these compositions do not always exhibit the gloss desired by consumers, in particular in the case of lipsticks.

The document US 2006/147390 teaches compositions comprising oils, waxes and a gas in a minimum amount of 20%. However, waxes alone do not make it possible to obtain a foam texture which is satisfactory and which exhibits an improved gloss.

A search is thus underway to obtain compositions which exhibit a normal texture, in the foam form, and which exhibit a satisfactory gloss, via the incorporation of a certain amount of oils which make it possible to contribute these gloss properties.

A search is also underway to formulate compositions comprising a high content of volatile or nonvolatile oils or organic solvents, so as to improve the hold and/or the resistance to water or to sebum and/or the transfer-free properties of the compositions.

In addition, the compositions in the foam form must exhibit good stability over time, in particular in terms of homogeneity and appearance of the product.

### SUMMARY OF THE INVENTION

It is one aim of the present invention to provide a formulation route for a composition for coating keratinous substances which exhibits a foam texture.

### DETAILED DESCRIPTION OF THE PREFERRED EMBODIMENTS

More specifically, one subject-matter of the invention is a composition in the form of a foam comprising a continuous oily phase and at least one structuring agent for the oily phase, the composition being devoid of wax.

The term “composition in (the) foam form” and the term “composition in the form of a foam” mean the same thing and are understood to mean a composition comprising a gas phase (for example air) in the form of bubbles; another equivalent term is “composition expanded in volume”.

The composition in the foam form preferably exhibits a light texture and is easy to withdraw and to spread over keratinous substances.

Another subject-matter of the invention is a method for coating keratinous substances, comprising the application, to the keratinous substances, of at least one layer of at least one composition in the foam form comprising a continuous oily phase and at least one structuring agent for the oily phase, the composition being devoid of wax.

One method for coating keratinous substances according to the invention comprises applying the composition in the foam form to keratinous substances; it differs from the processes of the prior art in that the foam is not formed in situ on the keratinous substances, that is to say that the foam is not created after application of the composition. In particular, a delayed-expansion composition, which is a system in which a “volatile” agent is released or formed in the composition after the latter has been applied to the keratinous substances, is not involved. Specifically, delayed-expansion compositions are created after exposure of a gel to atmospheric pressure and/or to shearing and/or to a temperature greater than ambient temperature.

Another subject-matter of the invention is a kit for making up and/or for a nontherapeutic care of keratinous substances, comprising:
a composition in the foam form comprising a continuous oily phase and at least one structuring agent for the oily phase, the composition being devoid of wax, and
an applicator comprising at least one application component configured in order to apply the composition to the keratinous substances.

### Density

The composition in the foam form preferably exhibits in particular a density (d_{foam}) of less than or equal to 0.95, preferably of less than or equal to 0.9, better still of less than or equal to 0.8. The density is preferably greater than or equal to 0.2 and better still greater than or equal to 0.3.

The density is measured according to the following protocol: a container, the volume Vo (cm³) of which is known with an accuracy of ±0.005 cm³ (Vo being of the order of 10 cm³), is weighed by means of a precision balance to ±0.00005 g. Its weight is recorded as Wo (g). This container is carefully filled with the foam until the container overflows. The surface of the container is then levelled with a straight blade in order to obtain a perfectly flat foam surface. The weight W (g) of the container filled with foam is then measured.

The density corresponds to the ratio of the density of the composition, calculated as follows:

$${ρ}_{v} ( g / {cm}^{3} ) = \frac{W - Wo}{Vo}$$

to the density of water (1 g/cm³).

The composition employed in the process according to the invention exhibits a density of less than or equal to 0.95, preferably of less than or equal to 0.9, better still of less than or equal to 0.8. Preferably, the density of the composition is greater than or equal to 0.2 and better still greater than or equal to 0.3.

### Degree of Expansion in Volume

The degree of expansion in volume corresponds to the amount of gas incorporated in the composition.

It can in particular be defined by the following formula:

$$Degree of expansion in volume = \frac{{d}_{before expansion in volume} - {d}_{foam}}{{d}_{foam}} \times 100$$

The density of the composition before expansion in volume is measured according to the method described above for the foam.

The composition in the foam form advantageously exhibits a degree of expansion in volume of greater than or equal to 10% and preferably of less than 350%, including 20, 30, 40, 50, 75, 100, 125, 150, 175, 200, 225, 250, 300, 325, etc. % and all values and subranges between stated values including, e.g., from 10% to 300%, preferably from 30% to 250% and better still from 40% to 200%.

### Size of the Bubbles:

As the composition according to the invention is in the foam form, it comprises gas bubbles, advantageously air bubbles. In particular, the air bubbles of the composition preferably exhibit a number-average diameter of less than or equal to 5 mm, for example ranging from 0.1 μm to 5 mm, preferably of less than or equal to 1 mm and better still of less than or equal to 0.8 mm, including, e.g., 0.7. 0.5. 0.3, etc. mm and all values and subranges between stated values.

The number-average diameter is determined in the following way: the foam is packaged in a pot as soon as it has finished being prepared, while it is still fluid. The size of the bubbles is observed by taking a digital photograph of the surface of the pot 24 h after manufacture or, after opening the packaging, after having levelled the surface, by then counting, over a surface area of approximately 1 cm², the number of bubbles using the “Saisam” image processing software and by determining their number-average diameter.

### Solids Content

The compositions according to the invention can advantageously exhibit a content of dry matter (or solids content) of, e.g., greater than or equal to 60% by weight, with respect to the total weight of the composition, preferably of greater than or equal to 65% by weight and better still of greater than or equal to 70% by weight, it being possible for the solids content to range up to 100% by weight, with respect to the total weight of the composition.

The content of dry matter, that is to say the content of nonvolatile matter, can be measured in various ways. Mention may be made, for example, of the methods by drying in an oven, the methods by drying by exposure to infrared radiation and the chemical methods by assaying the water according to the Karl Fischer method.

Preferably, the amount of dry matter, commonly referred to as “solids content”, of the compositions according to the invention is measured by heating the sample by infrared rays with a wavelength of 2 μm to 3.5 μm. The substances present in the compositions which have a high vapour pressure evaporate under the effect of this radiation. The measurement of the loss in weight of the sample makes it possible to determine “the solids content” of the composition. These measurements are carried out by means of a Mettler LP16 commercial infrared dryer. This technique is fully described in the documentation of the device supplied by Mettler.

The measurement protocol is as follows:

Approximately 1 g of the composition is spread over a metal dish. The latter, after introduction into the dryer, is subjected to a set temperature of 120° C. for one hour. The wet weight of the sample, corresponding to the starting weight, and the dry weight of the sample, corresponding to the weight after exposure to the radiation, are measured by means of a precision balance.

The content of dry matter is calculated in the following way:

Solids content=100×(dry weight/wet weight).

Preferably, the composition employed in the method according to the invention is a leave-in composition.

### I/Continuous Oily Phase

The composition according to the invention comprises a continuous oily phase (or liquid fatty phase).

The term “oily phase” is understood to mean, within the meaning of the patent application, a phase composed of one or more nonaqueous fatty substances which are liquid at ambient temperature (25° C.) and atmospheric pressure (760 mmHg), also known as oils or organic solvents, and which are compatible with one another.

The continuous oily phase can represent, e.g., from 10 to 95% of the total weight of the composition, preferably from 20 to 85% and better still from 40 to 80% by weight.

The oil can be chosen from volatile oils and/or nonvolatile oils, and their mixtures.

The term “composition comprising an oily continuous phase” is understood to mean that the composition exhibits a conductivity, measured at 25° C., of less than 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the two electrodes of the cell. The conductivity is read as soon as the value of the conductivity meter has stabilized. A mean is determined over at least three successive measurements.

The term “volatile oil” is understood to mean, within the meaning of the invention, an oil capable of evaporating on contact with the skin or the keratinous fibre in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40000 Pa (10⁻³ to 300 mmHg), in particular ranging from 1.3 Pa to 13000 Pa (0.01 to 100 mmHg) and more particularly ranging from 1.3 Pa to 1300 Pa (0.01 to 10 mmHg).

The term “nonvolatile oil” is understood to mean an oil which remains on the skin or the keratinous fibre at ambient temperature and atmospheric pressure for at least several hours and which has in particular a vapour pressure of less than 10⁻³ mmHg (0.13 Pa).

These oils can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur and phosphorus atoms.

Volatile hydrocarbon oils can be chosen from hydrocarbon oils having from 8 to 16 carbon atoms, in particular branched C8-C16 alkanes, such as C8-C16 isoalkanes of petroleum origin (also known as isoparaffins), such as isododecane (also known as 2,2,4,4,6-pentamethylheptane), isodecane or isohexa-decane, for example the oils sold under the Isopar or Permethyl trade names, branched C8-C16 esters, isohexyl neopentanoate, and their mixtures. Other volatile hydrocarbon oils, such as petroleum distillates, in particular those sold under the Shell Solt name by Shell, can also be used. According to one embodiment, the volatile solvent is chosen from volatile hydrocarbon oils having from 8 to 16 carbon atoms and their mixtures.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 7 silicon atoms, these silicones optionally comprising alkyl or alkoxy groups having from 1 to 10 carbon atoms. Mention may in particular be made, as volatile silicone oil which can be used in the invention, of octamethylcyclotetrasiloxane, decamethyl-cyclopentasiloxane, dodecamethylcyclohexasiloxane, heptamethylhexyltrisiloxane, heptamethyloctyltri-siloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I)

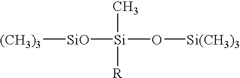

where R represents an alkyl group comprising from 2 to 4 carbon atoms, one or more hydrogen atoms of which can be substituted by a fluorine or chlorine atom.

Mention may be made, among the oils of general formula (I), of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is respectively a butyl group, a propyl group or an ethyl group.

Use may also be made of volatile fluorinated solvents, such as nonafluoromethoxybutane or perfluoromethyl-cyclopentane.

The composition can comprise at least one nonvolatile oil, chosen in particular from nonvolatile hydrocarbon oils and/or silicone oils and/or fluorinated oils.

Mention may in particular be made, as nonvolatile hydrocarbon oil, of:
hydrocarbon oils of vegetable origin, such as triesters of fatty acids and of glycerol, the fatty acids of which can have varied chain lengths from C4 to C24, it being possible for these chains to be linear or branched and saturated or unsaturated; these oils are in particular wheat germ, sunflower, grape seed, sesame, maize, apricot kernel, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or even triglycerides of caprylic/capric acids, such as those sold by Stéarineries Dubois or those sold under the names Miglyol 810, 812 and 818 by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam oil, squalane and their mixtures;
synthetic esters, such as the oils of formula R₁COOR₂ in which R₁, represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (cetostearyl octanoate), isopropyl myristate, isopropyl palmitate, C12 to C15 alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;
fatty alcohols comprising a branched and/or unsaturated carbon chain having from 12 to 26 carbon atoms which are liquid at ambient temperature, such as octyldodecanol, isostearyl alcohol, oleyl alcohol, 2-hexyldecanol, 2-butyloctanol or 2-undecylpentadecanol;
higher fatty acids, such as oleic acid, linoleic acid or linolenic acid;
carbonates;
acetals;
citrates;
and their mixtures.

The nonvolatile silicone oils which can be used in the composition according to the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethyl-siloxy)diphenylsiloxanes, diphenyl dimethicones, diphenyl(methyldiphenyl)trisiloxanes or (2-phenyl-ethyl)trimethylsiloxysilicates.

The fluorinated oils which can be used in the invention are in particular fluorosilicone oils, fluorinated polyethers or fluorinated silicones, such as disclosed in the document EP-A-847 752.

According to one embodiment, the fatty phase comprises a nonvolatile oil, preferably a hydrocarbon oil, in particular an ester oil. This ester oil can be chosen from esters of monocarboxylic acids with monoalcohols and polyalcohols.

Advantageously, the ester corresponds to the following formula (I):

R₁—CO—O—R₂ (I)

where R₁ represents a linear or branched alkyl radical of 1 to 40 carbon atoms, preferably of 7 to 19 carbon atoms, which optionally comprises one or more ethylenic double bonds and which is optionally substituted,
R₂ represents a linear or branched alkyl radical of 1 to 40 carbon atoms, preferably of 3 to 30 carbon atoms and better still of 3 to 20 carbon atoms which optionally comprises one or more ethylenic double bonds and which is optionally substituted.

The term “optionally substituted” is understood to mean that R₁ and/or R₂ can carry one or more substituents chosen, for example, from groups comprising one or more heteroatoms chosen from O, N and S, such as amino, amine, alkoxy or hydroxyl.

Preferably, the total number of carbon atoms of R₁+R₂ is greater than ≧9.

R₁ can represent the residue of a linear or, preferably, branched fatty acid, preferably a higher fatty acid, comprising from 1 to 40 and better still from 7 to 19 carbon atoms and R₂ can represent a linear or, preferably, branched hydrocarbon chain comprising from 1 to 40, preferably from 3 to 30 and better still from 3 to 20 carbon atoms. Again, preferably, the number of carbon atoms of R₁+R₂≧9.

Examples of R₁ groups are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, eleostearic acid, arachidonic acid, erucic acid and their mixtures.

Examples of esters are, for example, Purcellin oil (cetostearyl octanoate), isononyl isononanoate, isopropyl mystristate, 2-ethylhexyl palmitate, 2-octyl-dodecyl stearate, 2-octyldodecyl erucate, isostearyl isostearate and heptanoates, octanoates, decanoates or ricinoleates of alcohols or of polyalcohols, for example of fatty alcohols.

Advantageously, the esters are chosen from the compounds of the above formula (I) in which R₁, represents an unsubstituted linear or branched alkyl group optionally comprising one or more ethylenic double bonds and of 1 to 40 carbon atoms, preferably of 7 to 19 carbon atoms, and R₂ represents an unsubstituted linear or branched alkyl group optionally comprising one or more ethylenic double bonds and of 1 to 40 carbon atoms, preferably of 3 to 30 carbons and better still of 3 to 20 carbon atoms.

Preferably, R₁ is an unsubstituted branched alkyl group of 4 to 14 carbon atoms, preferably of 8 to 10 carbon atoms, and R₂ is an unsubstituted branched alkyl group of 5 to 15 carbon atoms, preferably of 9 to 11 carbon atoms. Preferably, in the formula (I), R₁—CO— and R₂ have the same number of carbon atoms and derive from the same radical, preferably an unsubstituted branched alkyl radical, for example the isononyl radical, that is to say that, advantageously, the ester oil molecule is symmetrical.

The ester oil will preferably be chosen from the following compounds:
isononyl isononanoate,
cetostearyl octanoate,
isopropyl myristate,
2-ethylhexyl palmitate,
2-octyldodecyl stearate,
2-octyldodecyl erucate,
isosteryl isostearate.

The oily phase can comprise from 0.5 to 100% by weight, in particular from 1 to 80% by weight, especially from 2 to 30% by weight and more particularly from 2 to 40% by weight of ester oil or oils.

### II/Structuring Agent

The structuring agent for the oily phase includes one or more such agents and is advantageously chosen from polymeric structuring agents.

The term “polymer” is understood to mean, within the meaning of the invention, a compound having at least 2 repeat units, preferably at least 3 repeat units and better still 10 repeat units.

The polymeric structuring agent advantageously exhibits a weight-average molecular weight of greater than or equal to 4000, better still of greater than or equal to 5000 and even better still of greater than or equal to 6000 which can range up to 500000.

The polymeric structuring agents can in particular be chosen from:
A) silicone polymers comprising at least one unit comprising:
   1) polyorganosiloxanes comprising at least two groups capable of establishing hydrogen interactions, these two groups being situated in the chain of the polymer, and/or
   2) polyorganosiloxanes comprising at least two groups capable of establishing hydrogen interactions, these two groups being situated on grafts or branchings,
   the groups capable of establishing hydrogen interactions being chosen from ester, amide, sulphonamide, carbamate, thiocarbamate, urea, urethane, thiourea, oxamido, guanidino and biguanidino groups and their combinations,
B) polyamide polymers comprising a polymeric backbone having hydrocarbon repeat units provided with at least one nonpendent amide unit and at least one optionally functionalized pendent fatty chain and/or at least one optionally functionalized end fatty chain which comprise at least 4 carbon atoms and which are bonded to these hydrocarbon units,
C) olefin copolymers,
D) partially or completely crosslinked organopolysiloxane elastomers of three-dimensional structure,
E) semicrystalline polymers,
F) esters of dextrin and of fatty acid, and their blends.

The structuring agent or agents can represent from 0.1 to 60% by weight, with respect to the total weight of the composition, preferably from 0.5 to 50% by weight and more preferably still from 1 to 40% by weight.

The amount of oily phase structuring agent can be adjusted by a person skilled in the art according to the structuring properties of the agents.

### A) Silicone Polymers

The silicone polymers of the composition are preferably solid at ambient temperature (25° C.) and atmospheric pressure (760 mmHg).

The term “polymer” is understood to mean, within the meaning of the invention, a compound having at least 2 repeat units, preferably at least 3 repeat units and better still 10 repeat units.

The silicone polymers used as structuring agents in the composition of the invention are polymers of the polyorganosiloxane type, such as, for example, those described in the documents U.S. Pat. No. 5,874,069, U.S. Pat. No. 5,919,441, U.S. Pat. No. 6,051,216 and U.S. Pat. No. 5,981,680.

According to the invention, the polymers used as structuring agent can belong to the following two families:
1) polyorganosiloxanes comprising at least two groups capable of establishing hydrogen interactions, these two groups being situated in the chain of the polymer, and/or
2) polyorganosiloxanes comprising at least two groups capable of establishing hydrogen interactions, these two groups being situated on grafts or branchings.

The groups capable of establishing hydrogen interactions can be chosen from ester, amide, sulphonamide, carbamate, thiocarbamate, urea, urethane, thiourea, oxamido, guanidino and biguanidino groups and their combinations.

a) According to a first alternative form, the silicone polymers are polyorganosiloxanes as defined above, the groups capable of establishing hydrogen interactions of which are positioned in the chain of the polymer.

The silicone polymers can more particularly be polymers comprising at least one unit corresponding to the general formula (I):

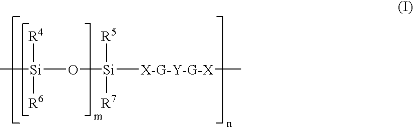

in which:
1) R⁴, R⁵, R⁶ and R⁷, which are identical or different, represent a group chosen from:
   saturated or unsaturated and linear, branched or cyclic C₁ to C₄₀ hydrocarbon groups which can comprise, in their chain, one or more oxygen, sulphur and/or nitrogen atoms and which can be substituted, partially or completely, by fluorine atoms,
   C₆ to C₁₀ aryl groups optionally substituted by one or more C1 to C4 alkyl groups,
   polyorganosiloxane chains comprising or not comprising one or more oxygen, sulphur and/or nitrogen atoms,
2) the X groups, which are identical or different, represent a linear or branched C₁ to C₃₀ alkylenediyl group which can comprise, in its chain, one or more oxygen and/or nitrogen atoms,
3) Y is a C₁ to C₅₀ alkylene (linear or branched), arylene, cycloalkylene, alkylarylene or arylalkylene divalent group, saturated or unsaturated, which can comprise one or more oxygen, sulphur and/or nitrogen atoms and/or carry, as substituent, one of the following atoms or groups of atoms: fluorine, hydroxyl, C₃ to C₈ cycloalkyl, C₁ to C₄₀ alkyl, C₅ to C₁₀ aryl, phenyl optionally substituted by 1 to 3 groups of the type C₁ to C3 alkyl, C1 to C3 hydroxyalkyl and C₁ to C6 aminoalkyl, or
4) Y represents a group corresponding to the formula:

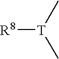

in which:
T represents a saturated or unsaturated and linear or branched trivalent or tetravalent C₃ to C₂₄ hydrocarbon group which is optionally substituted by a polyorganosiloxane chain and which can comprise one or more atoms chosen from O, N and S, or T represents a trivalent atom chosen from N, P and Al, and
R⁸ represents a linear or branched C₁ to C₅₀ alkyl group or a polyorganosiloxane chain, which can comprise one or more ester, amide, urethane, thiocarbamate, urea, thiourea and/or sulphonamide groups, which may or may not be bonded to another chain of the polymer,
5) the G groups, which are identical or different, represent divalent groups chosen from:

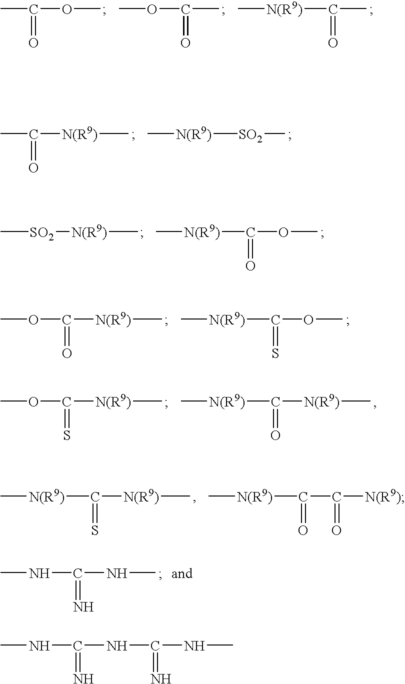

where R⁹ represents a hydrogen atom or a linear or branched C₁ to C₂₀ alkyl group, provided that at least 50% of the R⁹ groups of the polymer represent a hydrogen atom and that at least two of the G groups of the polymer are a group other than:

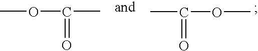

6) n is an integer ranging from 2 to 500, preferably from 2 to 200, and m is an integer ranging from 1 to 1000, preferably from 1 to 700 and better still from 6 to 200.

According to the invention, 80% of the R⁴, R⁵, R⁶ and R⁷ groups of the polymer are preferably chosen from methyl, ethyl, phenyl and 3,3,3-trifluoropropyl groups.

According to the invention Y can represent various divalent groups, furthermore optionally comprising one or two free valences in order to establish bonds with other units of the polymer or copolymer. Preferably, Y represents a group chosen from:
a) linear C₁ to C20, preferably C₁ to C₁₀, alkylene groups,
b) branched C₃₀ to C₅₆ alkylene groups which can comprise rings and nonconjugated unsaturations,
c) C₅-C₆ cycloalkylene groups,
d) phenylene groups optionally substituted by one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C1 to C3 hydroxyalkyl and C₁ to C6 alkylamine groups,
g) polyorganosiloxane chains of formula:

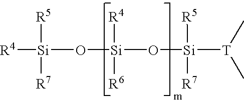

in which R⁴, R⁵, R⁶, R⁷, T and m are as defined above, and
h) polyorganosiloxane chains of formula:

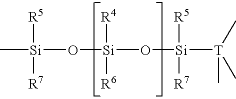

b) According to the second alternative form, the polyorganosiloxanes can be polymers comprising at least one unit corresponding to the formula (II):

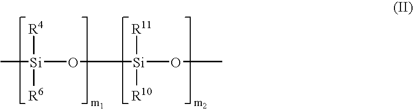

in which:
R⁴ and R⁶, which are identical or different, are as defined above for the formula (I),
R¹⁰ represents a group as defined above for R⁴ and R⁶, or represents the group of formula —X-G-R¹² in which X and G are as defined above for the formula (I) and R¹² represents a hydrogen atom or a saturated or unsaturated and linear, branched or cyclic C₁ to C₅₀ hydrocarbon group which optionally comprises, in its chain, one or more atoms chosen from O, S and N and which is optionally substituted by one or more fluorine atoms and/or one or more hydroxyl groups, or a phenyl group optionally substituted by one or more C1 to C4 alkyl groups,
R¹¹ represents the group of formula —X-G-R¹² in which X, G and R¹² are as defined above,
m₁ is an integer ranging from 1 to 998, and
m₂ is an integer ranging from 2 to 500.

According to the invention, the polymer used as structuring agent can be a homopolymer, that is to say a polymer comprising several identical units, in particular units of formula (I) or of formula (II).

According to the invention, use may also be made of a polymer composed of a copolymer comprising several different units of formula (I), that is to say a polymer in which at least one of the values R⁴, R⁵, R⁶, R⁷, X, G, Y, m and n is different in one of the units. The copolymer can also be formed from several units of formula (II) in which at least one of the values R⁴, R⁶, R¹⁰, R¹¹, m₁ and m₂ is different in at least one of the units.

Use may also be made of a polymer comprising at least one unit of formula (I) and at least one unit of formula (II), it being possible for the units of formula (I) and the units of formula (II) to be identical to or different from one another.

According to an alternative form of the invention, use may also be made of a polymer additionally comprising at least one hydrocarbon unit comprising two groups capable of establishing hydrogen interactions chosen from ester, amide, sulphonamide, carbamate, thiocarbamate, urea, urethane, thiourea, oxamido, guanidino and biguanidino groups and their combinations.

These polymers can be block polymers or grafted polymers.

According to an advantageous embodiment of the invention, the groups capable of establishing hydrogen interactions are amide groups of formulae —C(O)NH— and —HN—C(O)—.

In this case, the structuring agent can be a polymer comprising at least one unit of formula (III) or (IV):

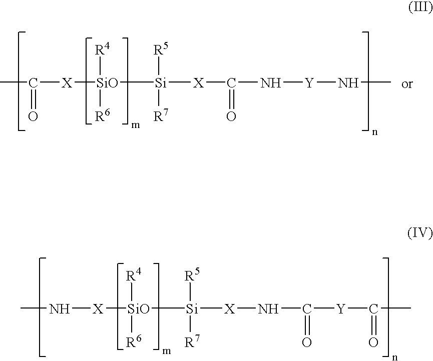

in which R⁴, R⁵, R⁶, R⁷, X, Y, m and n are as defined above.

Such a unit can be obtained:
either by a condensation reaction between a silicone comprising α,ω-carboxylic acid ends and one or more diamines, according to the following reaction scheme:

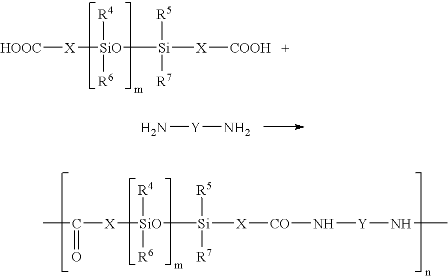

or by reaction of two molecules of α-unsaturated carboxylic acid with a diamine, according to the following reaction scheme:

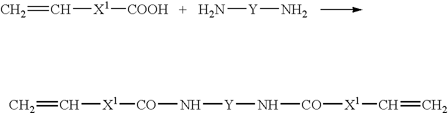

followed by the addition of a siloxane to the ethylenic unsaturations, according to the following scheme:

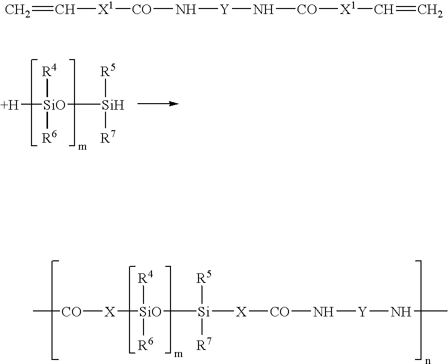

in which X¹—(CH₂)₂— corresponds to the X defined above and Y, R⁴, R⁵, R⁶, R⁷ and m are as defined above,
or by reaction of a silicone comprising α,ω-NH₂ ends and of a diacid of formula HOOC—Y—COOH, according to the following reaction scheme:

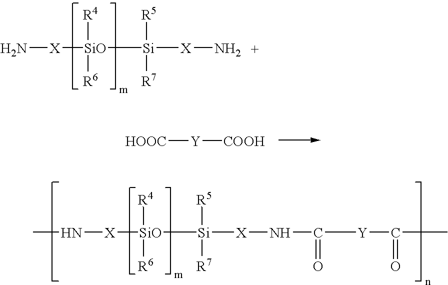

In these polyamides of formula (III) or (IV), m ranges from 1 to 700, in particular from 15 to 500 and especially from 50 to 200 and n ranges in particular from 1 to 500, preferably from 1 to 100 and better still from 4 to 25,
X is preferably a linear or branched alkylene chain having from 1 to 30 carbon atoms, in particular from 1 to 20 carbon atoms, especially from 5 to 15 carbon atoms and more particularly 10 carbon atoms, and
Y is preferably a linear or branched alkylene chain or an alkylene chain which can comprise rings and/or unsaturations having from 1 to 40 carbon atoms, in particular from 1 to 20 carbon atoms and better still from 2 to 6 carbon atoms, in particular 6 carbon atoms.

In the formulae (III) and (IV), the alkylene group representing X or Y can optionally comprise, in its alkylene part, at least one of the following components:
1) 1 to 5 amide, urea, urethane or carbamate groups,
2) a C5 or C6 cycloalkyl group, and
3) a phenylene group optionally substituted by 1 to 3 identical or different C1 to C3 alkyl groups.

In the formulae (III) and (IV), the alkylene groups can also be substituted by at least one component chosen from the group consisting of:
a hydroxyl group,
a C3 to CB cycloalkyl group,
one to three C₁ to C₄₀ alkyl groups,
a phenyl group optionally substituted by one to three C1 to C3 alkyl groups,
a C₁ to C3 hydroxyalkyl group, and
a C₁ to C6 aminoalkyl group.

In these formulae (III) and (IV), Y can also represent:

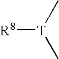

where R⁸ represents a polyorganosiloxane chain and T represents a group of formula:

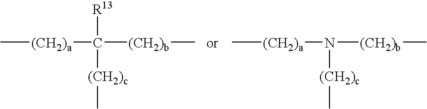

in which a, b and c are independently integers ranging from 1 to 10 and R¹³ is a hydrogen atom or a group such as those defined for R⁴, R⁵, R⁶ and R⁷.

In these formulae (III) and (IV), R⁴, R⁵, R⁶ and R⁷ preferably independently represent a linear or branched C₁ to C₄₀ alkyl group, preferably a CH₃, C₂H₅, n-C₃H₇ or isopropyl group, a polyorganosiloxane chain or a phenyl group optionally substituted by one to three methyl or ethyl groups.

As has been seen above, the polymer can comprise identical or different units of formula (III) or (IV).

Thus, the polymer can be a polyamide comprising several units of formula (III) or (IV) of different lengths or a polyamide corresponding to the formula (V):

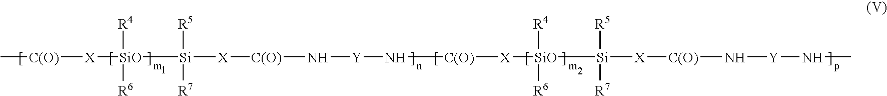

in which X, Y, n, R⁴ to R⁷ have the meanings given above, m₁ and m₂, which are different, are chosen within the range extending from 1 to 1000, and p is an integer ranging from 2 to 300.

In this formula, the units can be structured in order to form either a block copolymer or a random copolymer or an alternative copolymer. In this copolymer, the units may not only be of different lengths but may also have different chemical structures, for example having different Y groups. In this case, the polymer can correspond to the formula (VI):

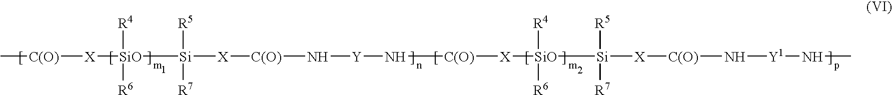

in which R⁴ to R⁷, X, Y, m₁, m₂, n and p have the meanings given above and Y¹ is other than Y but chosen from the groups defined for Y. As above, the different units can be structured in order to form either a block copolymer or a random copolymer or an alternating copolymer.

In this first embodiment of the invention, the structuring agent can also be composed of a grafted copolymer. Thus, the polyamide comprising silicone units can be grafted and optionally crosslinked by silicone chains comprising amide groups. Such polymers can be synthesized with trifunctional amines.

In this case, the polymer can comprise at least one unit of formula (VII):

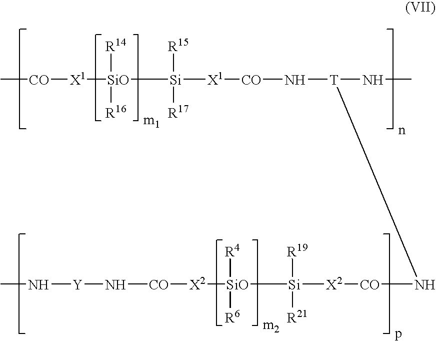

in which X¹ and X², which are identical or different, have the meaning given for X in the formula (I), n is as defined in the formula (I), Y and T are as defined in the formula (I), R¹⁴ to R²¹ are groups chosen from the same group as the groups R⁴ to R⁷, m₁ and m₂ are numbers lying within the range extending from 1 to 1000 and p is an integer ranging from 2 to 500.

In the formula (VII), it is preferable for:
p to range from 1 to 25, better still from 1 to 7,
R¹⁴ to R²¹ to be methyl groups,
T to correspond to one of the following formulae:

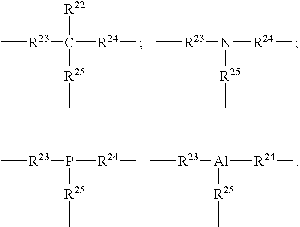

in which R²² is a hydrogen atom or a group chosen from the groups defined for R⁴ to R⁷, and R²³, R²⁴ and R²⁵ are independently linear or branched alkylene groups, more preferably with a formula:

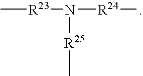

in particular with R²³, R²⁴ and R²⁵ representing —CH₂—CH₂—,
m₁ and m₂ to range from 15 to 500 and better still from 15 to 45,
X₁ and X₂ to represent —(CH₂)₁₀—, and
Y to represent —CH₂—.

These polyamides comprising a grafted silicone unit of formula (VII) can be copolymerized with silicone polyamides of formula (II) in order to form block copolymers, alternating copolymers or random copolymers. The percentage by weight of grafted silicone units (VII) in the copolymer can range from 0.5 to 30% by weight.

According to the invention, as has been seen above, the siloxane units can be in the main chain or backbone of the polymer but they can also be present in the grafted or pendent chains. In the main chain, the siloxane units can be in the form of segments as described above. In the pendent or grafted chains, the siloxane units can appear individually or in segments.

According to an alternative embodiment of the invention, use may be made of a copolymer of silicone polyamide and of hydrocarbon polyamide or of a copolymer comprising units of formula (III) or (IV) and hydrocarbon polyamide units. In this case, the silicone polyamide units can be positioned at the ends of the hydrocarbon polyamide.

According to a specific embodiment, the composition comprises at least one polydimethylsiloxane block polymer of general formula (I) having an index m with a value of approximately 100. More preferably, the composition according to the invention comprises at least one polymer comprising at least one unit of formula (III) where m varies from 50 to 600, in particular from 60 to 400, especially from 75 to 200 and more particularly is of the order of 100 and preferably again R⁴, R⁵, R⁶ and R⁷ independently represent, in the formula (III), a linear or branched C₁ to C₄₀ alkyl group, preferably a CH₃, C₂H₅, n-C₃H₇ or isopropyl group.

Preferably again, X and Y independently represent a group chosen from linear C₁ to C₂₀, preferably C₁ to C₁₀, alkylene groups.

Mention may be made, as examples of silicone polymer which can be used, of one of the silicone polyamides obtained in accordance with Examples 1 to 3 of the document U.S. Pat. No. 5,981,680.

According to an alternative embodiment of the invention, the polymer is composed of a homopolymer or copolymer comprising urethane or urea groups. These polymers are described in detail in Application WO 2003/106614, published on 24 Dec. 2003.

As above, such a polymer can comprise polyorganosiloxane units comprising two or more urethane and/or urea groups, either in the backbone of the polymer or on side chains or as pendent groups.

The polymers comprising at least two urethane and/or urea groups in the backbone can be polymer comprising at least one unit corresponding to the following formula (VIII):

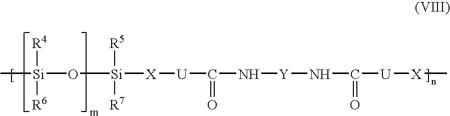

in which the R⁴, R⁵, R⁶, R⁷, X, Y, m and n symbols have the meanings given above for the formula (I) and U represents —O— or —NH—, in order for:

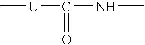

to correspond to a urethane or urea group.

In this formula (VIII), Y can be a linear or branched C₁ to C₄₀ alkylene group optionally substituted by a C₁ to C₁₅ alkyl group or a C₅ to C₁₀ aryl group. Preferably, use is made of a —(CH₂)₆— group.

Y can also represent a C₅ to C₁₂ cycloaliphatic or aromatic group which can be substituted by a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. It is generally preferable for Y to represent a linear or branched C₁ to C₄₀ alkylene radical or a C₄ to C₁₂ cycloalkylene radical.

Y can also represent a polyurethane or polyurea block corresponding to the condensation of several diisocyanate molecules with one or more molecules of couplers of the diol or diamine type. In this case, Y comprises several urethane or urea groups in the alkylene chain.

It can correspond to the formula (IX):

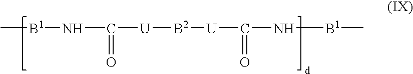

in which B¹ is a group chosen from the groups given above for Y, U is —O— or —NH— and B² is chosen from:
linear or branched C₁ to C₄₀ alkylene groups,
C₅ to C₁₂ cycloalkylene groups, optionally carrying alkyl substituents, for example one to three methyl or ethyl groups, or alkylene substituents, for example the radical of the diol: cyclohexanedimethanol,
phenylene groups which can optionally carry C1 to C3 alkyl substituents, and
groups of formula:

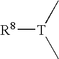

in which T is a trivalent hydrocarbon radical which can comprise one or more heteroatoms, such as oxygen, sulphur and nitrogen, and R⁸ is a polyorganosiloxane chain or a linear or branched C₁ to C₅₀ alkyl chain.

T can represent, for example:

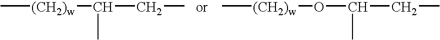

with w being an integer ranging from 1 to 10 and R⁸ being a polyorganosiloxane chain.

When Y is a linear or branched C₁ to C₄₀ alkylene group, the —(CH₂)₂— and —(CH₂)₆— groups are preferred.

In the formula given above for Y, d can be an integer ranging from 0 to 5, preferably from 0 to 3, more preferably equal to 1 or 2.

B² is preferably a linear or branched C₁ to C₄₀ alkylene group, especially —(CH₂)₂— or —(CH₂)₆—, or the group:

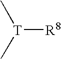

with R⁸ being a polyorganosiloxane chain.

As above, the silicone polymer can be formed of silicone-urethane and/or silicone-urea units with different lengths and/or constitutions and can be provided in the form of block or random (statistical) copolymers.

The polymers of formula (VIII) comprising urea or urethane groups in the chain of the silicone polymer can be obtained by reaction between a silicone comprising α,ω-NH₂ or —OH end groups of formula:

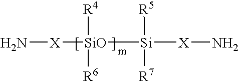

in which m, R⁴, R⁵, R⁶, R⁷ and X are as defined for the formula (I), and a diisocyanate OCN—Y—NCO where Y has the meaning given in the formula (I); and optionally a diol or diamine coupler of formula H₂N—B²—NH₂ or HO—B²—OH, where B² is as defined in the formula (IX).

Depending on the stoichiometric proportions between the two reactants, diisocyanate and coupler, it is possible to have, for Y, the formula (IX) with d equal to 0 or d equal to 1 to 5.

As in the case of the silicone polyamides of formula (IV), (II) or (III), use may be made, in the invention, of silicone polyurethanes or polyureas having units with different lengths and structures, in particular units which differ in length in the number of silicone units. In this case, the copolymer can correspond, for example, to the formula:

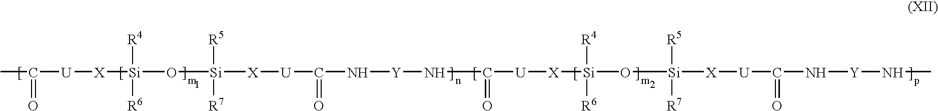

in which R⁴, R⁵, R⁶, R⁷, X, Y and U are as defined for the formula (VIII) and m₁, m₂, n and p are as defined for the formula (V).

According to the invention, the silicone can also comprise urethane and/or urea groups no longer in the backbone but in side branchings.

In this case, the polymer can comprise at least one unit of formula:

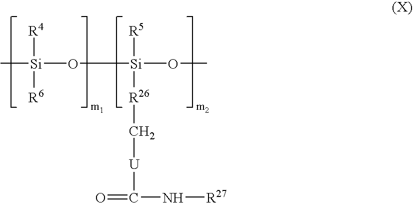

in which R⁴, R⁶, R⁵, m₁ and m₂ have the meanings given above for the formula (II) and R⁵ for the formula (I),
U represents O or NH,
R²⁶ represents a C₁ to C₄₀ alkylene group optionally comprising one or more heteroatoms chosen from O and N or a phenylene group, and
R²⁷ is chosen from saturated or unsaturated and linear, branched or cyclic C₁ to C₅₀ alkyl groups and from phenyl groups optionally substituted by one to three C1 to C3 alkyl groups.

The polymers comprising at least one unit of formula (X) comprise siloxane units and urea or urethane groups and they can be used as structuring polymer in the compositions of the invention.

The siloxane polymers can have a single urea or urethane group per branching or can have branchings comprising two urea or urethane groups or can also comprise a mixture of branchings comprising a urea or urethane group and or branchings comprising two urea or urethane groups.

They can be obtained from branched polysiloxanes comprising one or two amino groups per branching via reacting these polysiloxanes with monoisocyanates.

Mention may be made, as examples of starting polymers of this type having amino and diamino branchings, of the polymers corresponding to the following formulae:

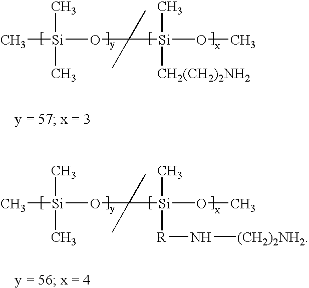

In these formulae, the symbol “/” in the case of the segments can be of different lengths and in any order and R represents a linear aliphatic group preferably having from 1 to 6 carbon atoms and better still from 1 to 3 carbon atoms.

Such polymers comprising a branching can be formed by reacting a siloxane polymer having at least three amino groups per polymer molecule with a compound having a single monofunctional group (for example an acid, an isocyanate or an isothiocyanate) in order to react this monofunctional group with one of the amino groups and to form the groups capable of establishing hydrogen interactions. The amino groups can be on side chains extending from the main chain of the siloxane polymer, so that the groups capable of establishing hydrogen interactions are formed on these side chains, or else the amino groups can be at the ends of the main chain, so that the groups capable of hydrogen interaction will be end groups of the polymer.

Mention may be made, as procedure for forming a polymer comprising siloxane units and groups capable of establishing hydrogen interactions, of the reaction of a siloxane diamine and of a diisocyanate in a silicone solvent, so as to directly form a gel. The reaction can be carried out in a silicone fluid, the reaction product being dissolved in this silicone fluid, at high temperature, the temperature of the system subsequently being reduced in order to form the gel.

The polymers preferred for the incorporation of the compositions of the present invention are siloxane-urea copolymers which are linear and which comprise urea groups as groups capable of establishing hydrogen interactions in the backbone of the polymer.

Mention may be made, by way of illustration of a polysiloxane terminated by four urea groups, of the polymer of formula:

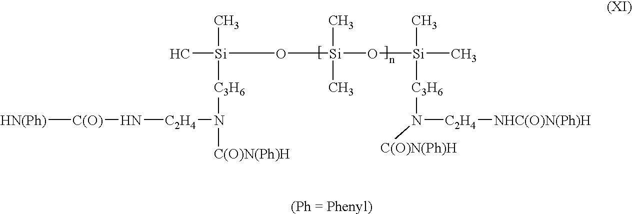

where Ph is a phenyl group and n is a number from 0 to 300, in particular from 0 to 100, for example 50.

This polymer is obtained by reaction of the following polysiloxane comprising amino groups:

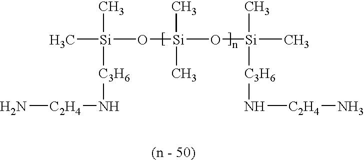

with phenyl isocyanate.

It is also possible to obtain branched silicone polyurethanes or polyureas by using, in place of the diisocyanate OCN—Y—NCO, a triisocyanate of formula:

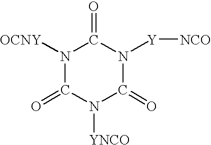

A silicone polyurethane or polyurea having branchings comprising an organosiloxane chain with groups capable of establishing hydrogen interactions is thus obtained. Such a polymer comprises, for example, a unit corresponding to the formula:

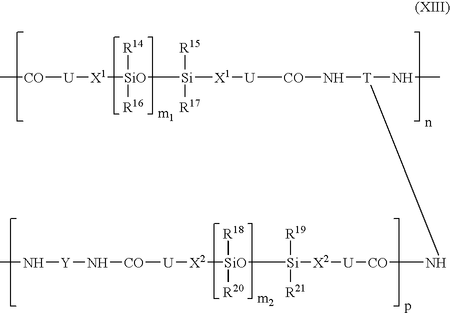

in which X¹ and X², which are identical or different, have the meaning given for X in the formula (I), n is as defined in the formula (I), Y and T are as defined in the formula (I), R¹⁴ to R²¹ are groups chosen from the same group as the R⁴ to R⁷ groups, m₁ and m₂ are numbers lying within the range extending from 1 to 1000 and p is an integer ranging from 2 to 500.

As in the case of the polyamides, use may be made, in the invention, of copolymers of silicone polyurethane, or polyurea, and of hydrocarbon polyurethane or polyurea by carrying out the reaction for the synthesis of the polymer in the presence of an α,ω-difunctional sequence of nonsilicone nature, for example a polyester, a polyether or a polyolefin.

As has been seen above, the copolymers of the invention can have siloxane units in the main chain of the polymer and groups capable of establishing hydrogen interactions either in the main chain of the polymer or at the ends of the latter, or on side chains or branchings of the main chain. This can correspond to the following five arrangements:

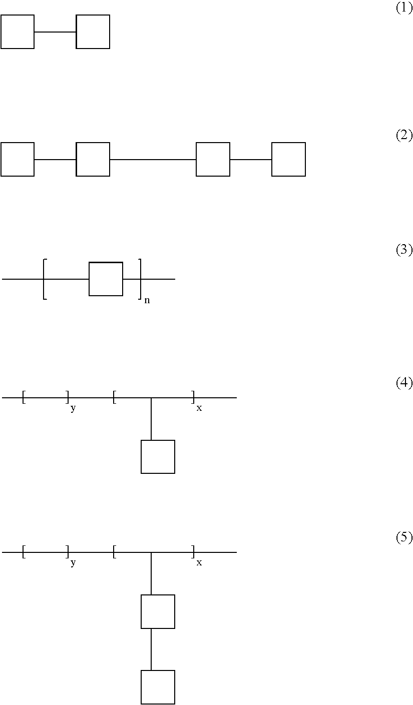

in which the continuous line is the main chain of the siloxane polymer and the squares represent the groups capable of establishing hydrogen interactions.

In the case (1), the groups capable of establishing hydrogen interactions are positioned at the ends of the main chain. In the case (2), two groups capable of establishing hydrogen interactions are positioned at each of the ends of the main chain.

In the case (3), the groups capable of establishing hydrogen interactions are positioned inside the main chain in repeat units.

In the cases (4) and (5), these are copolymers in which the groups capable of establishing hydrogen interactions are positioned on branchings of the main chain of a first series of units which are copolymerized with units not comprising groups capable of establishing hydrogen interactions.

The polymers and copolymers used in the composition of the invention advantageously have a solid/liquid transition temperature of 45° C. to 190° C. Preferably, they exhibit a solid/liquid transition temperature ranging from 70 to 130° C. and better still from 80° C. to 105° C.

These polymers can exhibit a weight-average molecular weight of 5000 to 500000, for example of 10000 to 300000, preferably of 20000 to 150000.

### B) Polyamide Polymers

As stated above, the polyamide polymer comprises a) a polymer backbone having hydrocarbon repeat units provided with at least one nonpendent amide unit and optionally b) at least one optionally functionalized pendent fatty chain and/or at least one optionally functionalized end fatty chain which comprise at least four carbon atoms and which are bonded to these hydrocarbon units.

The term “functionalized chains” is understood to mean, within the meaning of the invention, an alkyl chain comprising one or more functional or reactive groups chosen in particular from amide, hydroxyl, ether, oxyalkylene or polyoxyalkylene, halogen, including fluorinated or perfluorinated groups, ester, siloxane and polysiloxane groups. In addition, the hydrogen atoms of one or more fatty chains can be substituted at least partially by fluorine atoms.

The term “hydrocarbon repeat units” is understood to mean, within the meaning of the invention, a unit comprising from 2 to 80 carbon atoms and preferably from 2 to 60 carbon atoms, carrying hydrogen atoms and optionally oxygen atoms, which can be saturated or unsaturated and linear, branched or cyclic. These units each additionally comprise at least one amide group which is advantageously nonpendent and which occurs in the polymeric backbone.

Advantageously, the pendent chains are directly bonded to at least one of the nitrogen atoms of the polymeric backbone.

The polyamide can comprise, between the hydrocarbon units, silicone units or oxyalkylene units.

In addition, the polyamide of the composition of the invention advantageously comprises from 40 to 98% of fatty chains, with respect to the total number of the amide units and fatty chains and better still from 50 to 95%.

Preferably, the pendent fatty chains are bonded to at least one of the nitrogen atoms of the amide units of the polymer.

In particular, the fatty chains of this polyamide represent from 40 to 98% of the total number of the amide units and of the fatty chains and better still from 50 to 95%.

Advantageously, the polyamide exhibits a weight-average molecular weight of less than 100000 (in particular ranging from 1000 to 100000), especially of less than 50000 (in particular ranging from 1000 to 500000) and more particularly ranging from 1000 to 300000, preferably from 2000 to 20000 and better still from 2000 to 10000.

The polyamide is insoluble in water, in particular at 25° C. It especially does not comprise an ionic group.

Mention may be made, as preferred polyamides which can be used in the invention, of polyamides branched by pendent fatty chains and/or end fatty chains having from 6 to 120 carbon atoms and better still from 8 to 120 carbon atoms and in particular from 12 to 68 carbon atoms, each end fatty chain being bonded to the polyamide backbone via at least one bonding group. The bonding group can be chosen from ester, ether, amine, urea, urethane, thioester, thioether, thiourea and thiourethane groups. Preferably, these polymers comprise a fatty chain at each end of the polyamide backbone.

These polymers are preferably polymers resulting from a polycondensation between a dicarboxylic acid having at least 32 carbon atoms (having in particular from 32 to 44 carbon atoms) and an amine chosen from diamines having at least 2 carbon atoms (in particular from 2 to 36 carbon atoms) and triamines having at least two carbon atoms (in particular from 2 to 36 carbon atoms). The diacid is preferably a dimer resulting from a fatty acid comprising ethylene unsaturation having at least 16 carbon atoms, preferably from 16 to 24 carbon atoms, such as oleic acid, linoleic acid or linolenic acid. The diamine is preferably ethylenediamine, hexylenediamine or hexamethylenediamine. The triamine is, for example, ethylenetriamine. For the polymers comprising one or two end carboxylic acid groups, it is advantageous to esterify them with the monoalcohol having at least 4 carbon atoms, preferably from 10 to 36 carbon atoms and better still from 12 to 24 carbon atoms and even better still from 16 to 24 carbon atoms, for example 18 carbon atoms.

The polyamide of the composition according to the invention can be chosen in particular from polymers of following formula (A):

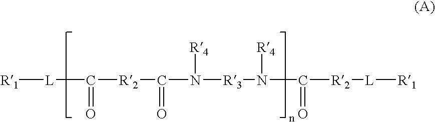

in which:
n is an integer ranging from 1 to 30,
R′₁ independently represents, in each case, a fatty chain and is chosen from an alkyl or alkenyl group having at least one carbon atom and in particular from 4 to 24 carbon atoms;
R′₂ independently represents, in each case, a hydrocarbon radical comprising from 1 to 52 carbon atoms;
R′₃ independently represents, in each case, an organic group comprising at least one atom chosen from carbon, hydrogen or nitrogen atoms, provided that R₁₃ comprises at least 3 carbon atoms;
R′₄ independently represents, in each case: a hydrogen atom, an alkyl group comprising from 1 to 10 carbon atoms or a direct bond to at least one group chosen from R′₃ and another R′₄, so that, when the group is another R′₄, the nitrogen atom to which both R′₃ and R′₄ are bonded forms part of a heterocyclic structure defined by R′₄—N—R′₃, provided that at least 50% of the R′₄ groups represent a hydrogen atom, and
L represents a bonding group as defined above in the description, optionally substituted by at least one R′₁ group as defined above.

According to one embodiment, these polymers are chosen from polymers of formula (A) in which the bonding group L represents an ester group

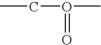

These polymers are more especially those described in the document U.S. Pat. No. 5,783,657 of Union Camp.

Each of these polymers satisfies in particular the following formula (B):

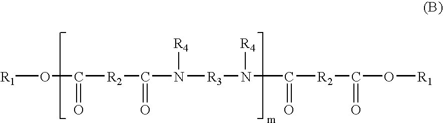

in which:
m denotes an integral number of amide units such that the number of ester groups represents from 10% to 50% of the total number of the ester and amide groups;
R₁ is in each case independently an alkyl or alkenyl group having at least 4 carbon atoms and in particular from 4 to 24 carbon atoms;
R₂ independently represents, in each case, a C₄ to C₄₂ hydrocarbon group, provided that 50% of the R₂ groups represent a C₃₀ to C₄₂ hydrocarbon group;
R₃ independently represents, in each case, an organic group provided with at least two carbon atoms, with hydrogen atoms and optionally with one or more oxygen or nitrogen atoms;
and R₄ independently represents, in each case, a hydrogen atom, a C₁ to C₁₀ alkyl group or a direct bond to R₃ or to another R₄, so that the nitrogen atom to which both R₃ and R₄ are bonded forms part of a heterocyclic structure defined by R₄—N—R₃, with at least 50% of the R₄ groups representing a hydrogen atom.

In the specific case of the formula (B), the optionally functionalized end fatty chains within the meaning of the invention are end chains bonded to the final nitrogen atom of the polyamide backbone.

In particular, the ester groups of the formula (B), which form part of the end and/or pendent fatty chains within the meaning of the invention, represent from 15 to 40% of the total number of ester and amide groups and better still from 20 to 35%.

Furthermore, m advantageously represents an integer ranging from 1 to 5 and better still of greater than 2.

Preferably, R₁ is a C12 to C22 and preferably C₁₆ to C₂₂ alkyl group.

Advantageously, R₂ can be a C10 to C₄₂ hydrocarbon (alkylene) group. Preferably, at least 50% and better still at least 75% of the R₂ groups are groups having from 30 to 42 carbon atoms. The other R₂ groups are hydrogenated C4 to C19 and even C4 to C12 groups.

Preferably, R₃ represents a C₂ to C₃₆ hydrocarbon group or a polyoxyalkylene group and R₄ represents a hydrogen atom. Preferably, R₃ represents a C₂ to C₁₂ hydrocarbon group.

The hydrocarbon groups can be saturated or unsaturated and linear, cyclic or branched groups. Furthermore, the alkyl and alkylene groups can be saturated or unsaturated and linear or branched groups.

Generally, the polymers of formula (B) are provided in the form of blends of polymers, it being possible for these blends to additionally comprise a synthetic product corresponding to a compound of formula (B) where m has the value of 0, that is to say a diester.

Mention may be made, as example of polyamides of formula (B) which can be used in the composition according to the invention, of the commercial products sold by Arizona Chemical under the names Uniclear 80 and Uniclear 100 VG, which are sold respectively in the form of an 80% (as active material) gel in a mineral oil and at 100% (as active material). They have a softening point from 88° C. to 94° C. These commercial products are a blend of copolymers of a C₃₆ diacid condensed with ethylenediamine, with a weight-average molecular weight of approximately 6000. The end ester groups result from the esterification of the remaining acid endings with cetyl alcohol, stearyl alcohol or their mixtures (also known as cetearyl alcohol).

Mention may also be made, as polyamides corresponding to the general formula (A), of polymers comprising at least one end fatty chain bonded to the polymeric backbone via at least one tertiary amide bonding group (also known as amide terminated polyamide or ATPA).

Such polymers are available, for example, under the reference Sylvaclear A 200 V from Arizona Chemical. For further information on these polymers, reference may be made to the document U.S. Pat. No. 6,503,522.

According to one embodiment, the polyamide of formula (A) can also comprise at least one end fatty chain bonded to the polymeric backbone via at least one ether or polyether bonding group (it is then said to be an ether terminated poly(ether)amide). Such polymers are described, for example, in the document U.S. Pat. No. 6,399,713.

Mention may also be made, as polyamide which can be used in the invention, of polyamide resins resulting from the condensation of an aliphatic dicarboxylic acid and of a diamine (including the compounds having more than 2 carbonyl groups and 2 amine groups), the carbonyl and amine groups of adjacent whole units being condensed via an amide bond. These polyamide resins are in particular those sold under the Versamid® trade mark by General Mills Inc. and Henkel Corp. (Versamid 930, 744 or 1655) or under the Onamid® trade mark, in particular Onamid S or C, by Olin Mathieson Chemical Corp. These resins have a weight-average molecular weight ranging from 6000 to 9000. For further information on these polyamides, reference may be made to the documents U.S. Pat. No. 3,645,705 and U.S. Pat. No. 3,148,125. More especially, Versamid® 930 or 744 is used.

It is also possible to use a poly(ester amide) comprising ester ends (ester-terminated poly(ester amide) or ETPEA), such as, for example, those whose preparation is described in the document U.S. Pat. No. 6,552,160. This polyamide is, for example, Sylvaclear C 75 V from Arizona Chemical.

It is also possible to use the polyamides sold by Arizona Chemical under the Uni-Rez references (2658, 2931, 2970, 2621, 2613, 2624, 2665, 1554, 2623, 2662) and the product sold under the reference Macromelt 6212 by Henkel. Such polymers are described, for example, in the document U.S. Pat. No. 5,500,209.

It is also possible to use polyamide resins resulting from vegetables, such as those described in U.S. Pat. No. 5,783,657 and U.S. Pat. No. 5,998,570.

The polyamide advantageously has a softening point of greater than 65° C. which can range up to 190° C. Preferably, it exhibits a softening point ranging from 70 to 130° C. and better still from 80 to 105° C. The polyamide is in particular a nonwaxy polymer.

Preferably, the polyamide corresponds to the formula (B) mentioned above.

### C) Olefin Copolymer

The composition according to the invention may comprise at least one olefin copolymer preferably chosen from amorphous olefin copolymers.

The term “olefin copolymer” is understood to mean, within the meaning of the present patent application, any copolymer formed by polymerization of at least one olefin and of another additional monomer other than the olefin.

The olefin can in particular be a monomer comprising ethylenic unsaturation.

Mention may be made, as example of olefin, of ethylenic hydrocarbon monomers having in particular one or two ethylenic unsaturations and having from 2 to 5 carbon atoms, such as ethylene, propylene, butadiene or isoprene.

The term “amorphous polymer” is understood to mean a polymer which does not have a crystalline form. This polymer can also be a film-forming polymer, that is to say that it is capable of forming a film when it is applied to the skin.

The olefin copolymer can in particular be a diblock, triblock, multiblock, radial or star copolymer or their blends.

Such compounds are described, for example, in Application US-A-2002/005562 and in U.S. Pat. No. 5,221,534.

An amorphous block copolymer of styrene and of olefin is advantageously chosen.

The block copolymer is preferably hydrogenated in order to reduce the residual ethylenic unsaturations after the polymerization of the monomers.

In particular, the hydrocarbon block copolymer is an optionally hydrogenated copolymer comprising styrene blocks and comprising ethylene/C₃ to C₄ alkylene blocks.

Mention may be made, as preferably hydrogenated diblock copolymer, of styrene-ethylene/propylene copolymers, styrene-ethylene/butadiene copolymers or styrene-ethylene/butylene copolymers. Diblock polymers are sold in particular under the name Kraton® G1701E by Kraton Polymers.

Mention may be made, as preferably hydrogenated triblock copolymers, of styrene-ethylene/propylene-styrene copolymers, styrene-ethylene/butadiene-styrene copolymers, styrene-isoprene-styrene copolymers or styrene-butadiene-styrene copolymers.

Triblock polymers are sold in particular under the names Kraton® G1650E, Kraton® G1652, Kraton® D1101, Kraton® D1102 and Kraton® D1160 by Kraton Polymers.

Use may in particular be made of a styrene-ethylene/butylene-styrene triblock copolymer.

According to one embodiment of the invention, use may in particular be made of a blend of a styrene-butylene/ethylene-styrene triblock copolymer and of a styrene-ethylene/butylene diblock copolymer sold in particular under the name Kraton® G1657M by Kraton Polymers.

Use may also be made of a blend of hydrogenated styrene-butylene/ethylene-styrene triblock copolymer and of hydrogenated ethylene-propylene-styrene star polymer, such a blend being in particular in isododecane. Such blends are sold, for example, by Penreco under the trade names Versagel® M5960 and Versagel® M5670.

### D) Organopolysiloxane Elastomers

The term “elastomer” is understood to mean a flexible and deformable material having viscoelastic properties and in particular the consistency of a sponge or of a flexible sphere. Its modulus of elasticity is such that this material is resistant to deformation and has a limited ability to expand and to contract. This material is capable of returning to its original shape after it has been stretched. This elastomer is formed of polymeric chains of high molecular weight, the mobility of which is limited by a uniform network of crosslinking points.

The organopolysiloxane elastomers used in the composition according to the invention are preferably solid and can be partially or completely crosslinked. When included in an oily phase, they are converted, according to the level of oily phase used, from a product with a spongy appearance, when they are used in the presence of low contents of oily phase, into a homogeneous gel in the presence of larger amounts of oily phase. The gelling of the oily phase by these elastomers can be complete or partial.

The elastomers of the invention can be conveyed in the form of a gel composed of an organopolysiloxane elastomer which includes at least one hydrocarbon oil and/or one silicone oil and/or one fluorinated oil. In addition, the oily phase associated with the organopolysiloxane elastomer can be composed of this oil or these oils.

The organopolysiloxane elastomers used according to the invention can be chosen from the crosslinked polymers described in Application EP-A-0295886. According to this patent application, they are obtained by an addition and crosslinking reaction, in the presence of a catalyst of the platinum type, of at least:
(a) one organopolysiloxane having at least two lower alkenyl groups per molecule, these alkenyl groups comprising from two to six carbon atoms; and
(b) one organopolysiloxane having at least two hydrogen atoms bonded to a silicone atom per molecule.

The organopolysiloxane elastomers used in the composition of the invention can also be chosen from those described in U.S. Pat. No. 5,266,321. According to this patent, they are chosen in particular from:
i) organopolysiloxanes comprising R₂SiO and RSiO_{1.5} units and optionally R₃SiO_{0.5} and/or SiO₂ units, in which the R radicals, independently of one another, represent a hydrogen, an alkyl radical, such as methyl, ethyl or propyl, an aryl radical, such as phenyl or tolyl, or an unsaturated aliphatic group, such as vinyl, the ratio by weight of the R₂SiO units to the RSiO_{1.5} units ranging from 1/1 to 30/1;
ii) organopolysiloxanes which are insoluble and which can be swollen in the silicone oil, obtained by addition of an organohydropolysiloxane (1) and of an organopolysiloxane (2) having unsaturated aliphatic groups, so that the amount of hydrogen or of unsaturated aliphatic groups in (1) and (2) respectively is between 1 and 20 mol % when the organopolysiloxane is not cyclic and between 1 and 50 mol % when the organopolysiloxane is cyclic.

The organopolysiloxane elastomers used in the composition of the invention can, for example, be those sold under the names KSG 6 by Shin-Etsu, Trefil E-505C or Trefil E-506C by Dow Corning or Gransil (SR-CYC, SR DMF10, SR-DC556) by Grant Industries, or those sold in the form of already constituted gels: KSG 15, KSG 16, KSG 17, KSG 18, KSG 26A, KSG 26B, KSG 41, KSG 42, KSG 43 and KSG 44 of Shin-Etsu, Gransil SR 5CYC Gel, Gransil SR DMF 10 Gel and Gransil SR DC556 Gel of Grant Industries, and 1229-02-167 and 1229-02-168 of General Electric. Use may also be made of a blend of silicone elastomers, in particular a blend of these commercial products.

### E) Semi-Crystalline Polymers

The term “polymer” is understood to mean compounds comprising at least two repeat units, preferably at least 3 repeat units and more especially at least 10 repeat units. The term “semicrystalline polymer” is understood to mean polymers comprising a crystallizable part, a crystallizable pendent chain or a crystallizable block in the backbone, and an amorphous part in the backbone and exhibiting a first-order reversible phase change temperature, in particular a melting point (solid-liquid transition). When the crystallizable part is in the form of a crystallizable block of the polymeric backbone, the amorphous part of the polymer is in the form of an amorphous block; in this case, the semicrystalline polymer is a block copolymer, for example of the diblock, triblock or multiblock type, comprising at least one crystallizable block and at least one amorphous block. The term “block” is generally understood to mean at least 5 identical repeat units. The crystallizable block or blocks are then different in chemical nature from the amorphous block or blocks.

The semicrystalline polymer has a melting point of greater than or equal to 30° C. (in particular ranging from 30° C. to 80° C.), preferably ranging from 30° C. to 60° C. This melting point is a first-order state change temperature.

This melting point can be measured by any known method and in particular using a differential scanning calorimeter (DSC).

Advantageously, the semicrystalline polymer or polymers to which the invention applies exhibits a number-average molecular weight of greater than or equal to 1000. Advantageously, the semicrystalline polymer or polymers of the composition of the invention have a number-average molecular weight Mn ranging from 2000 to 800000, preferably from 3000 to 500000, better still from 4000 to 150000, in particular of less than 100000 and better still from 4000 to 99000. Preferably, they exhibit a number-average molecular weight of greater than 5600, for example ranging from 5700 to 99000. The term “crystallizable chain or block” is understood to mean, within the meaning of the invention, a chain or block which, if it were alone, would change reversibly from the amorphous state to the crystalline state, according to whether the temperature is above or below the melting point. A chain within the meaning of the invention is a group of atoms which is in the pendent or side position with respect to the backbone of the polymer. A block is a group of atoms belonging to the backbone, a group constituting one of the repeat units of the polymer. Advantageously, the “crystallizable pendent chain” can be a chain comprising at least 6 carbon atoms.

The semicrystalline polymer can be chosen from block copolymers comprising at least one crystallizable block and at least one amorphous block, homopolymers and copolymers carrying at least one crystallizable side chain per repeat unit, and their blends.

Such polymers are described, for example, in the document EP 1 396 259.

According to a more particular embodiment of the invention, the polymer results from a monomer comprising a crystallizable chain chosen from saturated C₁₄ to C₂₂ alkyl (meth)acrylates.

Mention may be made, as specific example of structuring semicrystalline polymer which can be used in the composition according to the invention, of the Intelimer® products from Landec, described in the “Intelimer® polymers” brochure, Landec IP22 (Rev. 4-97). These polymers are in the solid form at ambient temperature (25° C.).

### F) Ester of Dextrin and of Fatty Acid(s)

The composition according to the invention preferably comprises at least one ester of dextrin and of fatty acid(s).

More particularly, it is a mono- or polyester of dextrin and of at least one fatty acid which corresponds in particular to the formula (C):

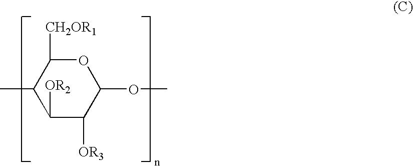

in which:
n is an integer ranging from 3 to 200, in particular ranging from 20 to 150 and especially ranging from 25 to 50,
the R₁, R₂ and R₃ radicals, which are identical or different, are chosen from hydrogen or an acyl group (R—CO—) in which the R radical is a saturated or unsaturated and linear or branched hydrocarbon group having from 5 to 29, in particular from 7 to 21, especially from 11 to 19, more particularly from 13 to 17, and even 15, carbon atoms, with the proviso that at least one of the R₁, R₂ or R₃ radicals is other than hydrogen.

In particular, R₁, R₂ and R₃ can represent hydrogen or an acyl group (R—CO—) in which R is a hydrocarbon radical as defined above, with the proviso that at least two of the R₁, R₂ or R₃ radicals are identical and other than hydrogen.

The combined R₁, R₂ and R₃ radicals can represent an acyl group (R—CO) which is identical or different and in particular identical.

In particular, n advantageously varies from 25 to 50 and in particular is equal to 38 in the general formula (C) of the ester according to the invention.

In particular when the R₁, R₂ and/or R₃ radicals, which are identical or different, represent an acyl group (R—CO), these can be chosen from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoaracic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic, stearolic, eicosanyl and docosanoyl radicals, and their mixtures.

Preferably, use is made, as ester of dextrin and of fatty acid(s), of at least a dextrin palmitate. The latter can be used alone or as a mixture with other esters.

Advantageously, the ester of dextrin and of fatty acid has a degree of substitution of less than or equal to 2.5 on the basis of one glucose unit, in particular varying from 1.5 to 2.5, preferably from 2 to 2.5. The weight-average molecular weight of the dextrin ester can in particular be from 10000 to 150000, in particular from 12000 to 100000 and indeed even from 15000 to 80000.

Dextrin esters, in particular dextrin palmitates, are available commercially under the name Rheopearl TL or Rheopearl KL from Chiba Flour.

### III) Additional Compounds

### Waxes

The composition according to the invention is devoid of wax.

The term “devoid of wax” is understood to mean a composition comprising less than 5% by weight, preferably less than 3% by weight, better still less than 2% by weight and even better still less than 1% by weight of wax, with respect to the total weight of the composition. More preferably, the composition does not comprise wax.

The wax under consideration in the context of the present invention is generally a lipophilic compound which is solid at ambient temperature (25° C.), which exhibits a reversible solid/liquid change in state and which has a melting point of greater than or equal to 30° C. which can range up to 200° C. and in particular up to 120° C.

On bringing the wax to the liquid state (melting), it is possible to render it miscible with oils and to form a microscopically homogeneous mixture but, on bringing the temperature of the mixture back to ambient temperature, recrystallization of the wax in the oils of the mixture is obtained.

In particular, the waxes can exhibit a melting point of greater than or equal to 45° C. and in particular of greater than or equal to 55° C.

Within the meaning of the invention, the melting point corresponds to the temperature of the most endothermic peak observed by thermal analysis (DSC) as described in the standard ISO 11357-3; 1999. The melting point of the wax can be measured using a differential scanning calorimeter (DSC), for example the calorimeter sold under the name “MDSC 2920” by TA Instruments.

The measurement protocol is as follows:

A 5 mg sample of wax placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

The waxes can be of animal, vegetable, mineral or synthetic origin, and their mixtures, which are solid at ambient temperature.

The waxes can generally exhibit a hardness ranging from 0.01 MPa to 15 MPa, in particular of greater than 0.05 MPa and especially of greater than 0.1 MPa.

The hardness is determined by the measurement of the compressive force measured at 20° C. using a texture analyser sold under the name TA-XT2 by Rheo, equipped with a stainless steel cylinder with a diameter of 2 mm which is displaced at the measuring rate of 0.1 mm/s and which penetrates the wax to a penetration depth of 0.3 mm.

The measurement protocol is as follows:

The wax is melted at a temperature equal to the melting point of the wax +10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least one hour before measuring the hardness or the tack.

The rotor of the texture analyser is displaced at a rate of 0.1 mm/s and then penetrates the wax to a penetration depth of 0.3 mm. When the rotor has penetrated the wax to the depth of 0.3 mm, the rotor is held stationary for 1 second (corresponding to the relaxation time) and is then withdrawn at the rate of 0.5 mm/s.

The value of the hardness is the maximum compressive force measured divided by the surface area of the cylinder of the texture analyser in contact with the wax.

The waxes can generally exhibit a weight-average molecular weight of less than or equal to 3000, better still of less than or equal to 2500 and even better still of less than or equal to 2000.

Mention may in particular be made, by way of illustration of waxes, of hydrocarbon waxes, such as beeswax, lanolin wax and Chinese insect waxes; rice bran wax, carnauba wax, candelilla wax, ouricury wax, esparto wax, berry wax, shellac wax, Japan wax and sumac wax; montan wax, orange and lemon waxes, microcrystalline waxes, paraffin waxes and ozokerite; polyethylene waxes, the waxes obtained by the Fischer-Tropsch synthesis and waxy copolymers, and their esters.

Mention may also be made of waxes obtained by catalytic hydrogenation of animal or vegetable oils having linear or branched C₈-C₃₂ fatty chains. Mention may in particular be made, among these, of isomerized jojoba oil, such as the transisomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane) tetrastearate, sold under the name of Hest 2T-4S® by Heterene.

Mention may also be made of silicone waxes or fluorinated waxes.

Use may also be made of the waxes obtained by hydrogenation of castor oil esterified with cetyl alcohol which are sold under the names of Phytowax Castor 16L64® and 22L73® by Sophim. Such waxes are described in Application FR-A-2 792 190.

Mention may also be made of waxes referred to as “tacky waxes”, that is to say having a tack of greater than or equal to 0.1 N.s and a hardness of less than or equal to 3.5 MPa.

The tacky wax used can have in particular a tack ranging from 0.1 N.s to 10 N.s, in particular ranging from 0.1 N.s to 5 N.s, preferably ranging from 0.2 to 5N.s and better still ranging from 0.3 to 2 N.s.

The tack of the wax is determined by the measurement of the change in the force (compressive force) as a function of the time at 20° C. according to the protocol indicated above for the hardness.

During the relaxation time of 1 s, the force (compressive force) strongly decreases until it becomes zero and then, during the withdrawal of the rotor, the force (stretching force) becomes negative to subsequently again increase towards the value 0. The tack corresponds to the integral of the curve of the force as a function of the time for the part of the curve corresponding to the negative values of the force. The value of the tack is expressed in N.s.

The tacky wax which can be used generally has a hardness of less than or equal to 3.5 MPa, in particular ranging from 0.01 MPa to 3.5 MPa, especially ranging from 0.05 MPa to 3 MPa.

Use may be made, as tacky wax, of a C₂₀-C₄₀ alkyl (hydroxystearyloxy)stearate (the alkyl group comprising from 20 to 40 carbon atoms), alone or as a mixture.

Such a wax is sold in particular under the names “Kester Wax K 82 P®”, “Hydroxypolyester K 82 P®” and “Kester Wax K 80 P®” by Koster Keunen.

### Film-Forming Polymer

The composition according to the invention can comprise at least one film-forming polymer.

The film-forming polymer can be present in the composition according to the invention in a content of dry matter (or active material) ranging from 0.1% to 30% by weight, with respect to the total weight of the composition, preferably from 0.5% to 20% by weight and better still from 1% to 15% by weight.

In the present invention, the term “film-forming polymer” is understood to mean a polymer capable of forming, by itself alone or in the presence of an additional agent which is able to form a film, a continuous film which adheres to a support, in particular to keratinous substances.

The composition according to the invention can comprise at least one film-forming polymer which is fat-soluble (that is to say, soluble in a liquid fatty phase comprising oils or organic solvents, such as described above) or lipophilic (that is to say, compatible with a liquid fatty phase comprising oils or organic solvents, such as those described above).

Mention may be made, as examples of fat-soluble polymer, of copolymers of vinyl ester (the vinyl group being directly connected to the oxygen atom of the ester group and the vinyl ester having a saturated and linear or branched hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated and linear or branched hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecane-dioate and divinyl octadecanedioate.

Mention may be made, as examples of these copolymers, of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of fat-soluble copolymers and in particular those resulting from the copolymerization of vinyl esters having from 9 to 22 carbon atoms or of alkyl acrylates or methacrylates, the alkyl radicals having from 10 to 20 carbon atoms.

Such fat-soluble copolymers can be chosen from copolymers of poly(vinyl stearate), of poly(vinyl stearate) crosslinked using divinylbenzene, diallyl ether or diallyl phthalate, copolymers of poly(stearyl (meth)acrylate), of poly(vinyl laurate), of poly(lauryl (meth)acrylate), it being possible for these poly(meth)acrylates to be crosslinked using ethylene glycol dimethacrylate or tetraethylene glycol dimethacrylate.

The fat-soluble copolymers defined above are known and are described in particular in Application FR-A-2 232 303; they can have a weight-average molecular weight ranging from 2000 to 500000 and preferably from 4000 to 200000.

Mention may also be made, as fat-soluble film-forming polymers which can be used in the invention, of polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (VP) and in particular copolymers of vinylpyrrolidone and of C₂ to C₄₀ alkene and better still C₃ to C₂₀ alkene. Mention may be made, as examples of VP copolymer which can be used in the invention, of the VP/vinyl acetate, VP/ethyl methacrylate, VP/ethyl meth-acrylate/methacrylic acid, VP/eicosene, VP/hexadecene, VP/triacontene, VP/styrene or VP/acrylic acid/lauryl methacrylate copolymer or butylated polyvinylpyrrolidone (PVP).

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

Mention may be made, as examples of commercially available polymethylsilsesquioxane resins, of those which are sold:
by Wacker under the reference Resin MK, such as Belsil PMS MK;
by Shin-Etsu under the reference KR-220L.

Mention may be made, as siloxysilicate resins, of trimethylsiloxysilicate (TMS) resins, such as those sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above, with polydimethylsiloxanes, such as the pressure-sensitive adhesive copolymers sold by Dow Corning under the reference BIO-PSA and described in the document U.S. Pat. No. 5,162,410 or the silicone copolymers resulting from the reaction of a silicone resin, such as those described above, and of a diorganosiloxane, such as are described in the document WO 2004/073626.

According to one embodiment of the invention, the film-forming polymer is a film-forming linear block ethylenic polymer which preferably comprises at least one first block and at least one second block having different glass transition temperatures (Tg), the first and second blocks being connected to one another via an intermediate block comprising at least one constituent monomer of the first block and at least one constituent monomer of the second block.

Advantageously, the first and second blocks of the block polymer are incompatible with one another.

Such polymers are described, for example, in the documents EP 1 411 069 or WO04/028488.

The lipophilic or fat-soluble film-forming polymer can also be present in the composition in the form of particles in dispersion in a nonaqueous solvent phase which can be that of the composition according to the invention. The techniques for the preparation of these dispersions are well known to a person skilled in the art.

Mention may be made, as examples of nonaqueous dispersions of film-forming polymer, of the dispersion described, for example, in the document EP 749 746 and in particular acrylic polymer particles, stabilized at the surface by a stabilizer, in dispersion in a fatty phase (for example isododecane), such as Mexomer PAP® from Chimex, or dispersions of particles of a grafted ethylenic polymer, preferably an acrylic polymer, in a liquid fatty phase, the ethylenic polymer advantageously being dispersed in the absence of additional stabilizer at the surface of the particles, such as described in particular in the document WO 04/055081.

The film-forming polymer can also be provided in the form of particles in dispersion in an aqueous phase, such as, for example, acrylic dispersions, sold under the names Neocryl XK-90®, Neocryl A-1070®, Neocryl A-1090®, Neocryl BT-62®, Neocryl A-1079® and Neocryl A-523® by Avencia Neoresins, Dow Latex 432® by Dow Chemical, Daitosol 5000 AD® or Daitosol 5000 SJ® by Daito Kasey Kogyo or Syntran 5760® by Interpolymer, or aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by Bayer, Aquamere H-1511® by Hydromer; sulphopolyesters, sold under the trade name Eastman AQ® by Eastman Chemical Products, vinyl dispersions, such as Mexomer PAM® from Chimex, and their blends.

The composition according to the invention can comprise a plasticizing agent favourable to the formation of a film with the film-forming polymer. Such a plasticizing agent can be chosen from any compound known to a person skilled in the art as being capable of fulfilling the desired role.

### Colouring Material

The composition according to the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or noncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type and lakes, based on cochineal carmine, of barium, strontium, calcium or aluminium.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annatto.

These colouring materials can be present in a content ranging from 0.01 to 30% by weight, with respect to the total weight of the composition.

### Fillers

The composition according to the invention can additionally comprise at least one filler.

The fillers can be chosen from those well known to a person skilled in the art and commonly used in cosmetic compositions. The fillers can be inorganic or organic and lamellar or spherical. Mention may be made of talc, mica, silica, kaolin, powders formed of polyamide, such as Nylon®, sold under the name Orgasol® by Atochem, of poly-β-alanine and of polyethylene, powders formed of tetrafluoroethylene polymers, such as Teflon®, lauroyllysine, starch, boron nitride, hollow polymer microspheres which are expanded, such as those of poly(vinylidene chloride)/acrylonitrile, for example those sold under the name Expancel® by Nobel Industrie, acrylic powders, such as those sold under the name Polytrap® by Dow Corning, particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms and in particular from 12 to 18 carbon atoms, for example zinc stearate, magnesium stearate, lithium stearate, zinc laurate or magnesium myristate.

Use may also be made of a compound capable of swelling when heated and in particular of heat-expandable particles, such as nonexpanded microspheres formed of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel.

The fillers can represent from 0.1 to 25% by weight, in particular from 1 to 20% by weight, with respect to the total weight of the composition.

### Aqueous Phase

The composition according to the invention can comprise water and/or water-soluble solvent(s) which can be introduced as such into the formulation according to the invention or can be incorporated therein via one or more ingredients constituting the composition. Thus, water can in particular be introduced into the composition via the introduction of a latex or pseudolatex, that is to say of an aqueous dispersion of polymer particles.

The water-miscible solvents (miscibility in water of greater than 50% by weight at 25° C.) are in particular lower monoalcohols having from 1 to 5 carbon atoms, such as ethanol or isopropanol, glycols having from 2 to 8 carbon atoms, such as propylene glycol, ethylene glycol, 1,3-butylene glycol or dipropylene glycol, C₃-C₄ ketones, C₂-C₄ aldehydes and their mixtures.

Advantageously, the composition exhibits a content of water and/or water-soluble solvent(s) of less than or equal to 15% by weight, with respect to the total weight of the composition, preferably of less than or equal to 10% by weight, better still of less than or equal to 5% by weight and even better still of less than or equal to 3% by weight.

According to one embodiment, the composition according to the invention is devoid of water (anhydrous composition).

### Fibres

The composition according to the invention can additionally comprise fibres which make possible an improvement in the lengthening effect.

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention can be fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury.

In particular, the fibres have a length ranging from 1 μm to 10 mm, in particular from 0.1 mm to 5 mm and more particularly from 0.3 mm to 3.5 mm. Their cross section can be included within a circle with a diameter ranging from 2 nm to 500 μm, in particular ranging from 100 nm to 100 μm and more particularly ranging from 1 μm to 50 μm. The weight or count of the fibres is often given in denier or decitex and represents the weight in grams per 9 km of yarn. The fibres according to the invention can in particular have a count chosen within the range from 0.15 to 30 denier and especially from 0.18 to 18 denier.

The fibres which can be used in the composition of the invention can be chosen from rigid or nonrigid fibres. They can be of synthetic or natural and inorganic or organic origin.

Furthermore, the fibres may or may not be surface treated, may or may not be coated and may or may not be coloured.

Mention may be made, as fibres which can be used in the composition according to the invention, of fibres which are not rigid, such as polyamide (Nylon®) fibres, or fibres which are rigid, such as polyimideamide fibres, for example those sold under the Kermel® or Kermel Tech® names by Rhodia, or poly(p-phenylene tereph-thalamide) (or aramid) fibres, sold in particular under the Kevlar® name by DuPont de Nemours.

The fibres can be present in the composition according to the invention in a content ranging from 0.01% to 10% by weight, with respect to the total weight of the composition, in particular from 0.1% to 5% by weight and more particularly from 0.3% to 3% by weight.

### Cosmetic Active Principles

Mention may in particular be made, as cosmetic active principles which can be used in the compositions according to the invention, of antioxidants, preservatives, fragrances, neutralizing agents, emollients, moisturizing agents, vitamins and screening agents, in particular sunscreens.

Of course, a person skilled in the art will take care to choose the optional additional additives and/or their amounts so that the advantageous properties of the composition according to the invention are not, or not substantially, detrimentally affected by the envisaged addition.

The composition before expansion in volume can be provided in the suspension, dispersion, solution or gel form.

### Preparation Process

The compositions employed in the invention can be prepared by processes for mixing, stirring or dispersing compressed gases, such as air, chlorofluorocarbon-based compounds, nitrogen, carbon dioxide, oxygen or helium, a process for mixing and stirring in the presence of a foaming agent, such as a surfactant.

In particular, the composition is prepared by mixing the ingredients with stirring, generally under hot conditions, and by then expanding in volume under the action of a gas, it being possible for the gas to be introduced during the stage of cooling the composition or after preparation of the composition, for example using a device for expanding in volume of Mondomix type, a beater of Kenwood type, a scraped-surface exchanger or a dynamic mixer (of IMT type, for example). The gas is preferably air or nitrogen.

The composition according to the invention can be packaged in a container delimiting at least one compartment which comprises the composition, the container being closed by a closure part. The container can be equipped with a means for the dispensing of the product. In particular, the container can be equipped with a pump.

The container can be a pot.

The container can be at least partly made of thermoplastic. Mention may be made, as examples of thermoplastics, of polypropylene or polyethylene. Alternatively, the container is made of nonthermoplastic material, in particular of glass or metal (or alloy).

The composition can be applied by finger or using an applicator.

The container is preferably used in combination with an applicator comprising at least one application component configured in order to apply the composition to keratinous substances.

According to another advantageous embodiment, the applicator comprises an application nozzle.

### EXAMPLES

The examples which follow are presented by way of illustration and without limitation of the invention. Unless otherwise indicated, the amounts are given as percent by weight.

### Examples 1 to 5

### Lipsticks in the Foam Form

The following lipstick compositions according to the invention are prepared:

| | | | | Exam- | Exam- |
|---|---|---|---|---|---|
| | Example 1 | Example 2 | Example 3 | ple 4 | ple 5 |
| Polyamide/poly- | 25 | 19.74 | 24.2 | | |
| dimethylsiloxane | | | | | |
| copolymer | | | | | |
| (n = 100) | | | | | |
| (DC 2-8179 from | | | | | |
| Dow Corning) | | | | | |
| Polyamide/poly- | | | | 10 | |
| dimethylsiloxane | | | | | |
| copolymer | | | | | |
| (n = 15) | | | | | |
| (DC 2-8178 from | | | | | |
| Dow Corning) | | | | | |
| Dextrin palmitate | | | | | 15 |
| (Rheopearl KL | | | | | |
| from Chiba Flour) | | | | | |
| Isononyl | q.s. for | q.s. for | q.s. | q.s. | q.s. |
| isononanoate | 100 | 100 | for 100 | for 100 | for 100 |
| Red 7 | 5 | 0.972 | 1.51 | 5 | 5 |
| Titanium dioxide | | 0.432 | 1.82 | | |
| Brown, yellow | | 2.052 | | | |
| iron oxide | | | | | |
| Black iron oxide | | | 1.36 | | |
| Yellow 5 lake | | 1.83 | | | |
| Red 27 lake | | | 1.26 | | |
| Yellow 6 lake | | | 2.05 | | |
| Blue 1 lake | | 0.432 | | | |
| Mica and titanium | | 6.042 | | | |
| dioxide | | | | | |
| Mica and titanium | | 1.08 | | | |
| dioxide | | | | | |
| Mica and titanium | | 2.16 | | | |
| dioxide | | | | | |

### Procedure

The oily structuring agent, the oil and the pigments are heated to 80° C. in a jacketed beaker with stirring of deflocculating paddle type.

The molten mixture is poured into a device for expanding in volume of “Mondomix” type using a peristaltic pump at a flow rate of 6 kg/h; the inlet temperature of the mixture displays 57° C. and the product is expanded in volume in the mixing head, which is thermostatically controlled at 20° C.

At the outlet of the device for expanding in volume, the foam, the temperature of which is 30° C., is packaged in pots which are immediately placed for 20 minutes in a chamber thermostatically controlled at −28° C.

For each of the compositions, the parameters of density and of degree of expansion in volume were measured according to the protocols described above.

The results are presented in the following table:

| | Example 1 | Example 2 | Example 3 | Example 4 | Example 5 |
|---|---|---|---|---|---|
| Density | 0.89 | 0.91 | 0.9 | 0.85 | 0.93 |
| before | | | | | |
| expanding in | | | | | |
| volume | | | | | |
| Density after | 0.52 | 0.39 | 0.33 | 0.7 | 0.73 |
| expanding in | | | | | |
| volume | | | | | |
| Degree of | 71% | 133% | 173% | 21% | 27% |
| expansion in | | | | | |
| volume | | | | | |

### Example 6

### Foundation

| | |
|---|---|
| Polyamide/polydimethysiloxane copolymer | 17.5 |
| (n = 100) | |
| (DC 2-8179 from Dow Corning) | |
| Isononyl isononanoate | Qsp 100 |
| Talc | 9.9 |
| Nylon powder | 9.9 |
| Vinylidene chloride/acrylonitrile/PMMA | 0.2 |
| copolymer microsphere (Expancel 551 DE20 | |
| D60 from Expancel) | |
| Titanium dioxide | 7.91 |
| Black iron oxide | 0.23 |
| Yellow iron oxide | 1.4 |
| Brown iron oxide | 0.46 |

The procedure used is the same as for the compositions of Examples 1 to 5 above.

The parameters of density and of degree of expansion in volume of the composition were measured according to the protocols described above.

| | |
|---|---|
| Density before expanding in volume | 0.97 |
| Density after expanding in volume | 0.52 |
| Degree of expansion in volume | 87% |

### Examples 7 to 10

### Colourless Compositions

The following compositions were prepared:

| | | | | Example |
|---|---|---|---|---|
| | Example 7 | Example 8 | Example 9 | 10 |
| Polyamide/poly- | 26.3 | 20 | 15 | 25 |
| dimethylsiloxane | | | | |
| copolymer | | | | |
| (n = 100) | | | | |
| (DC 2-8179 from Dow | | | | |
| Corning) | | | | |
| Isononyl isononanoate | 73.7 | 80 | 85 | |
| Dimethicone (DC Fluid | | | | 75 |
| 200, 5 cSt from Dow | | | | |
| Corning) | | | | |

The procedure used is the same as for the compositions of Examples 1 to 5 above.

The parameters of density and of degree of expansion in volume of each composition were measured according to the protocols described above.

| | | | | Example |
|---|---|---|---|---|
| | Example 7 | Example 8 | Example 9 | 10 |
| Density before | 0.9 | 0.89 | 0.88 | 0.95 |
| expanding in volume | | | | |
| Density after | 0.39 | 0.42 | 0.39 | 0.45 |
| expanding in volume | | | | |
| Degree of expansion | 131% | 112% | 126% | 111% |
| in volume | | | | |

### Examples 11 to 13

### Colourless Compositions

The following compositions were prepared:

| | Example | Example | Example |
|---|---|---|---|
| | 11 | 12 | 13 |
| Polyamide comprising | 20 | | |
| end ester groups | | | |
| (“Uniclear ® 100 V” from | | | |
| Arizona Chemical) | | | |
| Styrene/ethylene- | | | 5 |
| butylene/styrene | | | |
| triblock copolymer | | | |
| (Kraton G 1650 E from | | | |
| Kraton Polymers) | | | |
| Dextrin palmitate | | 20 | |
| (Rheopearl TL2 from | | | |
| Chiba Flour) | | | |
| Isononyl isononanoate | 80 | 80 | |
| Parleam oil | | | 95 |

The procedure used is the same as for the compositions of Examples 1 to 5 above.

The parameters of density and of degree of expansion in volume of each composition were measured according to the protocols described above.

| | Example | Example | Example |
|---|---|---|---|
| | 11 | 12 | 13 |
| Density before | 0.85 | 0.91 | 0.8 |
| expanding in volume | | | |
| Density after | 0.75 | 0.73 | 0.68 |
| expanding in volume | | | |
| Degree of expansion | 15% | 25% | 18% |
| in volume | | | |

### Examples 14 and 15

| | Example | Example |
|---|---|---|
| | 14 | 15 |
| Polyamide/polydimethyl- | 20 | 20 |
| siloxane copolymer | | |
| (n = 100) | | |
| (DC 2-8179 from Dow | | |
| Corning) | | |
| Polyamide/polydimethyl- | 5 | |
| siloxane copolymer | | |
| (n = 15) | | |
| (DC 2-8178 from Dow | | |
| Corning) | | |
| Dextrin palmitate | | 5 |
| (Rheopearl TL2 from | | |
| Chiba Flour) | | |
| Isononyl isononanoate | 75 | 75 |

The procedure used is the same as for the compositions of Examples 1 to 5 above.

The parameters of density and of degree of expansion in volume of each composition were measured according to the protocols described above.

| | Example | Example |
|---|---|---|
| | 14 | 15 |
| Density before | 0.91 | 0.92 |
| expanding in volume | | |
| Density after | 0.40 | 0.37 |
| expanding in volume | | |
| Degree of expansion | 128% | 163% |
| in volume | | |

The above written description of the invention provides a manner and process of making and using it such that any person skilled in this art is enabled to make and use the same, this enablement being provided in particular for the subject matter of the appended claims, which make up a part of the original description and including a composition in the foam form comprising a continuous oily phase and at least one structuring agent for the oily phase, the composition being devoid of wax. The claims below are the measure of the scope of the invention and set out the required components.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition in the form of a foam comprising a continuous oily phase and at least one structuring agent for the oily phase, the composition being devoid of wax.

2. The composition according to claim 1, wherein it exhibits a density of less than or equal to 0.95.

3. The composition according to claim 1, wherein it exhibits a density of greater than or equal to 0.2.

4. The composition according to claim 1, wherein it exhibits a degree of expansion in volume ranging from 10% to 300%.

5. The composition according to claim 1, wherein the structuring agent or agents for the oily phase are present in a content ranging from 0.1 to 60% by weight, with respect to the total weight of the composition.

6. The composition according to claim 1, wherein the structuring agent for the oily phase is chosen from:
A) silicone polymers comprising at least one unit comprising:
1) polyorganosiloxanes comprising at least two groups capable of establishing hydrogen interactions, these two groups being situated in the chain of the polymer, and/or
2) polyorganosiloxanes comprising at least two groups capable of establishing hydrogen interactions, these two groups being situated on grafts or branchings,
the groups capable of establishing hydrogen interactions being chosen from ester, amide, sulphonamide, carbamate, thiocarbamate, urea, urethane, thiourea, oxamido, guanidino and biguanidino groups and their combinations,
B) polyamide polymers comprising a) a polymeric backbone having hydrocarbon repeat units provided with at least one nonpendent amide unit and, optionally, b) at least one optionally functionalized pendent fatty chain and/or at least one optionally functionalized end fatty chain which comprise at least 4 carbon atoms and which are bonded to these hydrocarbon units,
C) olefin copolymers,
D) organopolysiloxane elastomers,
E) semicrystalline polymers,
F) esters of dextrin and of fatty acid,
and their blends.

7. The composition according to claim 6, in which the structuring agent comprises at least one silicone polymer comprising at least one unit corresponding to the formula: 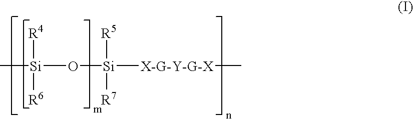
in which:
1) R⁴, R⁵, R⁶ and R⁷, which are identical or different, represent a group chosen from:
saturated or unsaturated and linear, branched or cyclic C₁ to C₄₀ hydrocarbon groups which can comprise, in their chain, one or more oxygen, sulphur and/or nitrogen atoms and which can be substituted, partially or completely, by fluorine atoms,
C₆ to C₁₀ aryl groups optionally substituted by one or more C1 to C4 alkyl groups,
polyorganosiloxane chains comprising or not comprising one or more oxygen, sulphur and/or nitrogen atoms,
2) the X groups, which are identical or different, represent a linear or branched C₁ to C₃₀ alkylenediyl group which can comprise, in its chain, one or more oxygen and/or nitrogen atoms,
3) Y is a C₁ to C₅₀ alkylene (linear or branched), arylene, cycloalkylene, alkylarylene or arylalkylene divalent group, saturated or unsaturated, which can comprise one or more oxygen, sulphur and/or nitrogen atoms and/or carry, as substituent, one of the following atoms or groups of atoms: fluorine, hydroxyl, C₃ to C₈ cycloalkyl, C₁ to C₄₀ alkyl, C₅ to C₁₀ aryl, phenyl optionally substituted by 1 to 3 groups of the type C1 to C3 alkyl, C1 to C3 hydroxyalkyl and C1 to C6 aminoalkyl, or
4) Y represents a group corresponding to the formula: 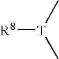
in which:
T represents a saturated or unsaturated and linear or branched trivalent or tetravalent C₃ to C₂₄ hydrocarbon group which is optionally substituted by a polyorganosiloxane chain and which can comprise one or more atoms chosen from O, N and S, or T represents a trivalent atom chosen from N, P and Al, and
R⁸ represents a linear or branched C₁ to C₅₀ alkyl group or a polyorganosiloxane chain, which can comprise one or more ester, amide, urethane, thiocarbamate, urea, thiourea and/or sulphonamide groups, which may or may not be bonded to another chain of the polymer,
5) the G groups, which are identical or different, represent divalent groups chosen from: 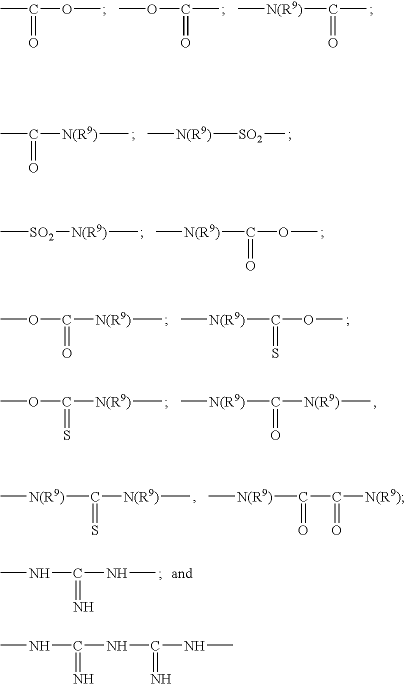
where R⁹ represents a hydrogen atom or a linear or branched C₁ to C₂₀ alkyl group, provided that at least 50% of the R⁹ groups of the polymer represent a hydrogen atom and that at least two of the G groups of the polymer are a group other than: 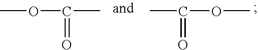
6) n is an integer ranging from 2 to 500, preferably from 2 to 200, and m is an integer ranging from 1 to 1000, preferably from 1 to 700 and better still from 6 to 200.

8. The composition according to claim 7, in which the polymer comprises at least one unit of formula (III) or (IV): 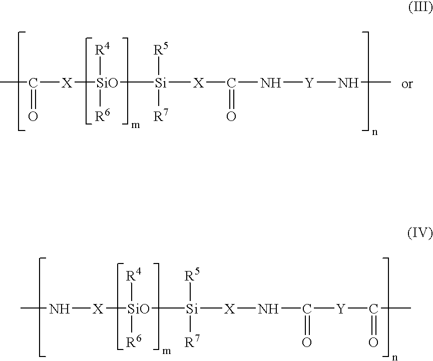
in which R⁴, R⁵, R⁶, R⁷, X, Y, m and n are as defined in claim 7.

9. The composition according to claim 6, in which the structuring agent comprises at least one polyamide chosen from polyamides of following formula (A): 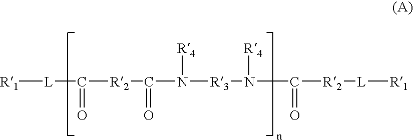
in which:
n is an integer ranging from 1 to 30,
R′₁ independently represents, in each case, a fatty chain and is chosen from an alkyl or alkenyl group having at least 1 carbon atom and in particular from 4 to 24 carbon atoms;
R′₂ independently represents, in each case, a hydrocarbon radical comprising from 1 to 52 carbon atoms;
R′₃ independently represents, in each case, an organic group comprising at least one atom chosen from carbon, hydrogen or nitrogen atoms, provided that R′₃ comprises at least 3 carbon atoms;
R′₄ independently represents, in each case: a hydrogen atom, an alkyl group comprising from 1 to 10 carbon atoms or a direct bond to at least one group chosen from R′₃ and another R′₄, so that, when the group is another R′₄, the nitrogen atom to which both R′₃ and R′₄ are bonded forms part of a heterocyclic structure defined by R′₄—N—R′₃, provided that at least 50% of the R′₄ groups represent a hydrogen atom, and
L represents a bonding group chosen from ester, ether, amine, urea, urethane, thioester, thioether, thiourea and thiourethane groups, optionally substituted by at least one R′₁ group.

10. The composition according to claim 9, wherein the polyamide is chosen from polyamides of following formula (B): 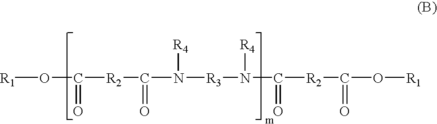
in which:
m denotes an integral number of amide units such that the number of ester groups represents from 10% to 50% of the total number of the ester and amide groups;
R₁ is in each case independently an alkyl or alkenyl group having at least 4 carbon atoms and in particular from 4 to 24 carbon atoms;
R₂ independently represents, in each case, a C₄ to C₄₂ hydrocarbon group, provided that 50% of the R₂ groups represent a C₃₀ to C₄₂ hydrocarbon group;
R₃ independently represents, in each case, an organic group provided with at least 2 carbon atoms, with hydrogen atoms and optionally with one or more oxygen or nitrogen atoms; and
R₄ independently represents, in each case, a hydrogen atom, a C₁ to C₁₀ alkyl group or a direct bond to R₃ or to another R₄, so that the nitrogen atom to which both R₃ and R₄ are bonded forms part of a heterocyclic structure defined by R₄—N—R₃, with at least 50% of the R₄ groups representing a hydrogen atom.

11. The composition according to claim 6, in which the structuring agent comprises at least one copolymer chosen from copolymers formed by a polymerization of styrene and of olefin, the olefin being chosen from ethylene, propylene, butadiene or isoprene.

12. The composition according to claim 11, wherein the olefin copolymer is chosen from optionally hydrogenated copolymers comprising styrene blocks and comprising ethylene/C₃-C₄ alkylene blocks.

13. The composition according to claim 6, in which the structuring agent comprises at least one ester of dextrin and of fatty acid(s) corresponding to the formula (C): 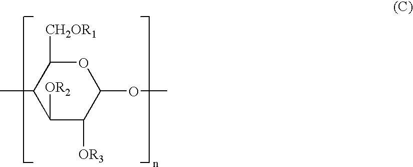
in which:
n is an integer ranging from 3 to 200,
the R₁, R₂ and R₃ radicals, which are identical or different, are chosen from hydrogen or an acyl group (R—CO—) in which the R radical is a saturated or unsaturated and linear or branched hydrocarbon group having from 5 to 29 carbon atoms, with the proviso that at least one of the R₁, R₂ or R₃ radicals is other than hydrogen.

14. The composition according to claim 13, wherein the ester of dextrin and of fatty acid(s) comprises at least dextrin palmitate.

15. The composition according to claim 1, wherein the continuous oily phase comprises at least one volatile oil. and nonvolatile oils.

16. The composition according to claim 1, wherein the continuous oily phase comprises at least one nonvolatile oil.

17. The composition according to claim 1, wherein it is anhydrous.

18. The composition according to claim 1, wherein it is not a delayed-expansion composition.

19. A method for coating a keratinous substance, comprising applying the composition according to claim 1 to the keratinous substance.

20. A kit, comprising:
a container comprising a composition in the foam form exhibiting a continuous oily phase and at least one structuring agent for the oily phase, the composition being devoid of wax, and
an applicator.
